(19) United States
(12) Patent Application Publication
(10) Pub. No.: US 2022/0252619 A1
(43) Pub. Date: Aug. 11, 2022

(54) DETECTION OF MEDIATORS OF DOPAMINE TRANSMISSION

(71) Applicant: UNIVERSITY OF FLORIDA RESEARCH FOUNDATION, INCORPORATED, Gainesville, FL (US)

(72) Inventor: Habibeh KHOSHBOUEI, Gainesville, FL (US)

(21) Appl. No.: 17/630,321

(22) PCT Filed: Jul. 27, 2020
(86) PCT No.: PCT/US2020/043784

§ 371 Date: Jan. 26, 2022

Related U.S. Application Data
(60) Provisional application No. 62/878,867, filed on Jul. 26, 2019.

(51) Int. Cl. G01N 33/68; G01N 1/30

(52) U.S. Cl. CPC G01N 33/6896; G01N 2800/2835; G01N 1/30

(57) ABSTRACT

Provided herein are novel assays for detecting mediators of dopamine transmission. Specifically exemplified, are assays for detecting tyrosine hydroxylase in human monocytes. Also disclosed is the use of assays for detecting or diagnosing Parkinson's disease or severity thereof.

### STATEMENT REGARDING FEDERALLY SPONSORED RESEARCH OR DEVELOPMENT

This invention was made with government support under Grant Nos. NS071122 and NS103108 awarded by the National Institutes of Health. The government has certain rights in the invention.

### BACKGROUND

The dysregulation of dopamine signaling and dopamine transporter is implicated in several neurodegenerative and neurocognitive pathologies, including Parkinson's disease and addiction. The dopamine transporter (DAT) and tyrosine hydroxylase (TH) regulate dopamine signaling in all cells expressing these two proteins. Increasing evidence suggests neurodegenerative disease states are associated with peripheral immune dysfunction, possibly impairing dopamine neurotransmission.

Peripheral immune cells of both lymphoid and myeloid lineage express known markers of the dopamine system, including proteins for synthesis, storage and release of dopamine, as well as dopamine transporter for dopamine clearance after signaling (5, 10, 13). Two mediators of dopamine transmission, dopamine transporter (DAT) and tyrosine hydroxylase (TH) are constitutively expressed on human monocytes (7, 10, 13). TH, the rate limiting enzyme in dopamine synthesis, and DAT, responsible for dopamine uptake, are biologically relevant targets to study dopamine transmission in these peripheral immune cells. Recent reports suggest that in neurodegenerative diseases, such as Parkinson's diseases, dopamine transmission is altered in both the CNS and the periphery (4,9). While immunohistochemical and immunofluorescent techniques have been widely used to determine DAT and TH expression in the CNS (3, 4), blood-borne immune cells present a novel milieu to study dopamine transmission. To-date, immunocytochemistry and qPCR assays have been used to determine TH and DAT expression in cultured monocytes (7, 10). These studies have provided valuable information about DAT and TH proteins or DNA expression; however, there is no high-throughput and quantifiable methodology available to determine the levels of these proteins acutely in fresh peripheral blood samples without culturing PMBCs that is likely to affect the endogenous levels of these proteins. Importantly, there are large repositories of PBMCs at different research institutes including NINDS PDBP and Michael J Fox Foundation's international PPMI initiative, and it is unknown if the existing preservation methodologies employed to preserve these samples alter the ratio of DAT and TH expressing PBMCs. Therefore, there is an unmet need to identify a cryopreservation methodology suitable for detection of DAT and TH in human monocytes.

Flow cytometry was originally developed for immunological detection of both surface and intracellular marker proteins to study immune cell populations in health and disease. In cell suspensions of blood-borne immune cells 12 or more markers can be distinguished depending on the capability of the flow cytometer in use

### BRIEF DESCRIPTION OF THE FIGURES

Many aspects of this disclosure can be better understood with reference to the following figures, in which:

FIG. 1A: is an example according to various embodiments illustrating representative plots showing the gating strategy for DAT-expressing and TH-expressing human monocytes by flow cytometry, and more specifically doublet discrimination and debris exclusion;

FIG. 1B: is an example according to various embodiments illustrating representative plots showing the gating strategy_for DAT- and TH-expressing human monocytes by flow cytometry, and more specifically demonstrating that DAT and TH expressing monocytes may be discriminated based on sidescatter properties intrinsic to monocytes (B, 1^{st} and 2^{nd} panel from left) or by expression of human monocyte marker CD14 (B, 3rd and 4th panel from left);

FIG. 1C: is an example according to various embodiments illustrating representative plots showing the gating strategy_for DAT- and TH-expressing human monocytes by flow cytometry, and more specifically demonstrating that isotype controls reveal minimal nonspecific staining;

FIG. 1D: is an example according to various embodiments illustrating representative plots showing the gating strategy_for DAT- and TH-expressing human monocytes by flow cytometry, and more specifically histogram representations for anti-DAT, anti-TH and anti-CD14 displaying mean fluorescence intensity (MFI) for: unstained (black), isotype control (blue) and immune-stained (red). N=41 independent biological replicates;

FIG. 2A: is an example according to various embodiments illustrating freshly isolated PBMCs show DAT, TH and CD14 expressing monocytes comparable to baseline for healthy individuals;

FIG. 2B: is an example according to various embodiments illustrating cryopreserved in FBSDMSO and quick-thawed PBMCs isolated from the same donor show dramatically altered percentage of PBMCs expressing DAT, TH and CD14;

FIG. 2C: is an example according to various embodiments illustrating cryopreserved PBMCs in FBSDMSO show clear CD14 staining, but a significant reduction of CD16 signal;

FIG. 2D is an example according to various embodiments illustrating altered percentage of DAT and TH expressing PBMCs before and after freeze-thaw;

FIG. 2E: is an example according to various embodiments illustrating significantly altered percentage of CD14+ to CD16+ monocytes shows the consequences of cryopreservation;

FIG. 3A: is an example according to various embodiments illustrating freshly isolated PBMCs show DAT, TH and CD14 expressing monocytes comparable to baseline for healthy individuals;

FIG. 3B: is an example according to various embodiments illustrating cryopreserved in FBS-DMSO and quick-thawed PBMCs isolated from the same donor show dramatically altered percentage of PBMCs expressing DAT, TH and CD14;

FIG. 3C: is an example according to various embodiments illustrating cryopreserved PBMCs in FBS-DMSO show clear CD14 staining, but a significant reduction of CD16 signal;

FIG. 3D: is an example according to various embodiments illustrating altered percentage of DAT and TH expressing PBMCs before and after freeze-thaw;

FIG. 3E: is an example according to various embodiments illustrating significantly altered percentage of CD14+ to CD16+ monocytes shows the consequences of cryopreservation;

FIGS. 4A, 4B, 4C, 4D, and 4E are examples according to various embodiments illustrating that serum-free cryopreservation maintains/preserve DAT/TH detection in monocyte subsets via representative plots from a statistical analysis conducted with computer software for analyzing and graphing scientific data, specifically GRAPHPAD PRISM® software, using multiple t tests with Holm-Sidak correction for multiple comparisons; alpha=0.05. N=3 independent biological replicates.

FIG. 4A: is an example according to various embodiments illustrating a representative plot of freshly isolated PBMCs;

FIG. 4B: is an example according to various embodiments illustrating a representative plot of serum-free cryopreservation and quick-thawed PBMCs isolated from the same donor maintains the percentage DAT+, TH+ and CD14+ PBMCs relative to fresh isolation;

FIG. 4C: is an example according to various embodiments illustrating a representative plot showing cryopreserved PBMCs show clear CD14 staining and slightly reduced CD16 signal relative to fresh isolation;

FIG. 4D: is an example according to various embodiments illustrating a representative plot of similar percentage of DAT and TH expressing PBMCs before and after serum-free freeze-thaw procedure; and

FIG. 4E: is an example according to various embodiments illustrating a representative plot showing there is a small, but not significant effect of cryopreservation using serum-free freezing media CryoStor10 on the percentage of CD14+ to CD16+ monocytes.

FIGS. 5A, 5B, 5C, 5D, and 5E are examples according to various embodiments illustrating a fluorescence-minus-one acquisition template setup of an FMO gating template to confirm constitutive DAT and TH expression in CD14+ monocyte.

FIG. 5A: is an example according to various embodiments illustrating sample lacking anti-CD14-FITC staining produces no events in DAT or TH gates;

FIG. 5B: is an example according to various embodiments illustrating a sample lacking anti-TH-BV421 produces no events in DAT/TH double positive gate;

FIG. 5C: is an example according to various embodiments illustrating a sample lacking anti-DAT-APC produces no DAT/TH double positive events;

FIG. 5D: is an example according to various embodiments illustrating an unstained sample according to FIG. 5E;

FIG. 5E: is an example according to various embodiments illustrating the sample of FIG. 5D stained with anti-CD14, anti-DAT and anti-TH to produce a triple positive population, demonstrating a stringently controlled gating strategy to confirm constitutive DAT and TH expression in human monocytes;

FIG. 6: is an example according to various embodiments illustrating an FMO gating template confirming specific detection of CD14 and CD16 with absence of spectral overlap and more specifically a fluorescence-minus-one acquisition template setup, including FMO samples used to establish positive gates for CD14 and CD16 expressing monocytes: from left to right, Unstained, CD14 only, CD16 only, and representative plot containing antibodies against CD14 and CD16;

FIG. 7: is an example according to various embodiments illustrating alternate anti-CD14 antibody clones and concentrations considered for this panel. Dilutions are in microliters of stock antibody as provided by the vendor, added to the final staining volume. Staining index (SI) was calculated using mean fluorescence index (MFI) and the formula: SI−[(MFI of positive cells)−(MFI of negative cells)]/(2*SD of negative cells).

FIGS. 8A, 8B, and 8C are examples according to various embodiments illustrating sample volunteer data demonstrating sensitivity of DAT and TH detection in human PBMCs at varied expression levels.

FIG. 8A: is an example according to various embodiments illustrating representative TH expression data from three independent healthy volunteers exhibiting varied levels of DAT and TH expressing monocytes;

FIG. 8B: is an example according to various embodiments illustrating representative DAT expression data from three independent healthy volunteers exhibiting varied levels of DAT and TH expressing monocytes;

FIG. 8C: is an example according to various embodiments illustrating representative CD14 expression data from three independent healthy volunteers exhibiting varied levels of DAT and TH expressing monocytes;

FIG. 9: is an example according to various embodiments, illustrating monocytes identified by side scatter were analyzed for TH/DAT expression as percentage of total PBMCs; alpha=0.05, one-way ANOVA with Tukey's test for multiple comparisons for whole blood acquired from PD patients and healthy, age-matched controls separated into PBMC fractions, and stained with antibodies against DAT and TH followed by appropriate secondary; and

FIG. 10: is an example according to various embodiments, illustrating results for mouse monocytes isolated from gradual depletion mice treated with 6-OHDA or saline (control group) via intracranial cannula targeted to the median-forebrain bundle, and assayed for expression of DAT and TH via flow cytometry.

FIGS. 11A, 11B, 11C, 11D, 11E, 11F, and 11G are examples according to various embodiments illustrating that an ELISA embodiment reproducibly and quantitatively detects tyrosine hydroxylase.

FIG. 11A: shows a diagram depicting the expression and purification of TH.

FIG. 11B: shows a western blot demonstration that TH is detectable in both purified construct and PC12 cells using rabbit polyclonal antibodies.

FIG. 11C: shows a western blot demonstration that TH is detectable in both purified construct and PC12 cells using mouse monoclonal antibodies.

FIG. 11D: shows a western blot demonstration that TH is detectable in both purified construct and PC12 cells using rabbit polyclonal antibodies.

FIG. 11E: depicts detection scheme using an HRP-conjugate secondary.

FIG. 11F: depicts detection scheme using a tertiary layer using anti-rabbit biotin followed by Avidin-HRP.

FIG. 11G: depicts detection scheme using a biotinylated detection antibody followed by avidin-HRP.

FIGS. 12A, 12B, and 12C, are examples according to various embodiments validating that an ELISA embodiment reproducibly and quantitatively detects tyrosine hydroxylase in cultured human macrophages.

FIG. 12A: shows a bar graph demonstrating TH levels (pgTH/mg total protein) in PC12 cells, HEK293 cells, human macrophages, and mouse dopaminergic neurons.

FIG. 12B: shows an absorbance graph demonstrating ng/mL of TH in PC12 cells, HEK293 cells, human macrophages, and mouse dopaminergic neurons.

FIG. 12C: provides a table of raw data related to graphs of FIGS. 12A and 12B.

FIGS. 13A, 13B, and 13C, are examples according to various embodiments validating that an ELISA embodiment reproducibly and quantitatively detects tyrosine hydroxylase in monocytes from healthy and Parkinson's patients. The data shows that TH levels are increased in monocytes isolated from Parkinson's disease patients.

FIG. 13A: shows a bar graph demonstrating TH levels (pgTH/mg total protein) in monocytes from healthy and Parkinson's patients.

FIG. 13B: shows an absorbance graph demonstrating ng/mL of TH in monocytes from healthy and Parkinson's patients.

FIG. 13C: provides a table of raw data related to graphs of FIGS. 13A and 13B.

FIG. 14 provides a diagram of a point of care device for detecting levels of a dopamine transmission mediator.

It should be understood that the various embodiments are not limited to the examples illustrated in the figures.

### DETAILED DESCRIPTION

### Introduction and Definitions

Various embodiments may be understood more readily by reference to the following detailed description. Unless defined otherwise, all technical and scientific terms used herein have the same meaning as commonly understood by one of ordinary skill in the art to which this disclosure belongs.

The terms “administering” or “administer” or “administration” as used herein with respect to an agent means providing the agent to a subject using any of the various methods or delivery systems for administering agents or pharmaceutical compositions known to those skilled in the art. Modes of administering include, but are not limited to oral administration, parenteral administration such as intravenous, subcutaneous, intramuscular or intraperitoneal injections, rectal administration by way of suppositories, transdermal administration, intraocular administration or administration by any route or method that delivers a therapeutically effective amount of the drug or composition to the cells or tissue to which it is targeted. Alternatively, routine experimentation will determine other acceptable routes of administration.

The terms “subject,” “individual,” “host,” and “patient,” are used interchangeably herein to refer to an animal being treated with one or more enumerated agents as taught herein, including, but not limited to, simians, humans, avians, felines, canines, equines, rodents, bovines, porcines, ovines, caprines, mammalian farm animals, mammalian sport animals, and mammalian pets. A suitable subject for the invention can be any animal, preferably a human, that is suspected of having, has been diagnosed as having, or is at risk of developing a disease that can be ameliorated, treated or prevented by administration of one or more enumerated agents.

The term “treating” or “treatment of” as used herein refers to providing any type of medical management to a subject. Treating includes, but is not limited to, administering a composition comprising one or more active agents to a subject using any known method for purposes such as curing, reversing, alleviating, reducing the severity of, inhibiting the progression of, or reducing the likelihood of a disease, disorder, or condition or one or more symptoms or manifestations of a disease, disorder or condition.

A “therapeutically effective amount” refers to an amount which, when administered in a proper dosing regimen, is sufficient to reduce or ameliorate the severity, duration, or progression of the disorder being treated (e.g., Parkinson's disease), prevent the advancement of the disorder being treated (e.g., Parkinson's disease), cause the regression of the disorder being treated (e.g., Parkinson's disease), or enhance or improve the prophylactic or therapeutic effects(s) of another therapy. The full therapeutic effect does not necessarily occur by administration of one dose and may occur only after administration of a series of doses. Thus, a therapeutically effective amount may be administered in one or more administrations per day for successive days.

Parkinson's therapy comprises a therapeutically effective amount of a composition comprising an agent known to treat Parknson's disease. Examples of Parkinson's therapy comprising administering therapeutically effective amounts of levodopa, carbidopa, a dopamine agonist, a MAO B inhibitor, a COMT inhibitor, an anticholinergic, or amantadine, or a combination thereof.

As used herein, the term “standard temperature and pressure” generally refers to 25° C. and 1 atmosphere. Standard temperature and pressure may also be referred to as “ambient conditions.” Unless indicated otherwise, parts are by weight, temperature is in ° C., and pressure is at or near atmospheric. The terms “elevated temperatures” or “high-temperatures” generally refer to temperatures of at least 100° C.

The term “mol percent” or “mole percent” generally refers to the percentage that the moles of a particular component are of the total moles that are in a mixture. The sum of the mole fractions for each component in a solution is equal to 1.

It is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by prior disclosure. Further, the dates of publication provided could be different from the actual publication dates that may need to be independently confirmed.

Unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

All the features disclosed in this specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

The examples and embodiments described herein are for illustrative purposes only and that various modifications or changes in light thereof will be suggested to persons skilled in the art and are to be included within the spirit and purview of this application. Many variations and modifications may be made to the above-described embodiment(s) of the disclosure without departing substantially from the spirit and principles of the disclosure. All such modifications and variations are intended to be included herein within the scope of this disclosure.

General Discussion

Building on traditional immunostaining protocols, various embodiments described herein establish a highly sensitive and reproducible multi-parameter flow cytometry method that can detect and quantify DAT and TH expression in peripheral blood monocytes of humans. After establishing a reproducible protocol in the freshly prepared samples, various embodiments compared the suitability of two different cryopreservation methods for detection of DAT and TH on peripheral monocytes. The side-by-side comparison revealed cryopreservation in FBS-DMSO media alters the immune phenotype of monocyte subsets, whereas cryopreservation in CryoStor10 media maintains the sample integrity. Various embodiments provide a methodology that provides a highly sensitive and quantifiable approach to study dopamine signaling in peripheral immune cells in healthy condition, in disease states with dysregulation of dopamine signaling, and following therapeutic interventions that directly or indirectly affect DAT or TH levels in the PBMCs.

Human peripheral immune cells, such as monocytes and macrophages, may express DAT and TH, but prior to various embodiments provided herein no high throughput methodology existed to determine DAT and TH expression in the peripheral immune cells. Various embodiments provide a highly sensitive, reproducible and novel approach using flow cytometry to detect DAT and TH expression in human peripheral blood monocytes. Since human peripheral blood mononuclear cells (PBMC) are routinely shared between laboratories, various embodiments identified an optimum cryopreservation method that unlike the traditional cryopreservation does not affect the integrity of cryopreserved PBMCs. Therefore, the detection and long-term storage methodologies according to various embodiments provide a sensitive and reproducible approach to examine dopamine signaling in peripheral human immune cells. These methodologies can be applied to study peripheral dopamine signaling under healthy and potentially under disease conditions. The use of dopamine signaling could also be explored as a technique to monitor therapeutic interventions particularly those targeting DAT and TH in the periphery.

Human monocytes express known markers of dopamine synthesis, storage and clearance, including dopamine transporter (DAT), tyrosine hydroxylase (TH), all subtypes of dopamine receptors and vesicular monoamine transporter 2 (VMAT2). Immunohistochemical and immunofluorescent methodologies have traditionally been employed to determine DAT and TH expression in the CNS, their detection in the blood and specifically in the peripheral monocytes has not been studied by flow cytometry. Flow cytometry assays are widely used in medicine and in basic, preclinical or clinical research to quantify physical and chemical characteristics of target cell populations. Various embodiments provide a highly sensitive and reproducible flow cytometry panel to detect and quantify DAT and TH expression in freshly isolated or cryopreserved human peripheral monocytes. In healthy humans (n=41 biological replicates), results according to various embodiments show baseline DAT and TH expressing monocytes constitute ˜12% of the peripheral blood mononuclear cell (PBMC) fraction when examined in fresh isolation from whole blood. Using an identical flow cytometry panel, results according to various embodiments demonstrate that cryopreservation of PBMCs using multiple techniques resulted in altered PBMC populations as compared to fresh isolation and relative to one another. Among these, results according to various embodiments identified an optimum cryopreservation method for detecting TH and DAT in cryopreserved PBMCs. Data obtained according to various embodiments provides a sensitive and reproducible approach to examine dopamine signaling in peripheral human immune cells. This approach can be applied to study peripheral dopamine signaling under healthy and potentially under disease conditions. The use of dopamine signaling could also be explored as a technique to monitor therapeutic interventions particularly those targeting DAT and TH in the periphery.

DAT and TH expressing monocytes increased in both Parkinson's patients and 6-OHDA gradual depletion mouse model, indicating mechanistic link between CNS dopaminergic degeneration and changes in peripheral immunity.

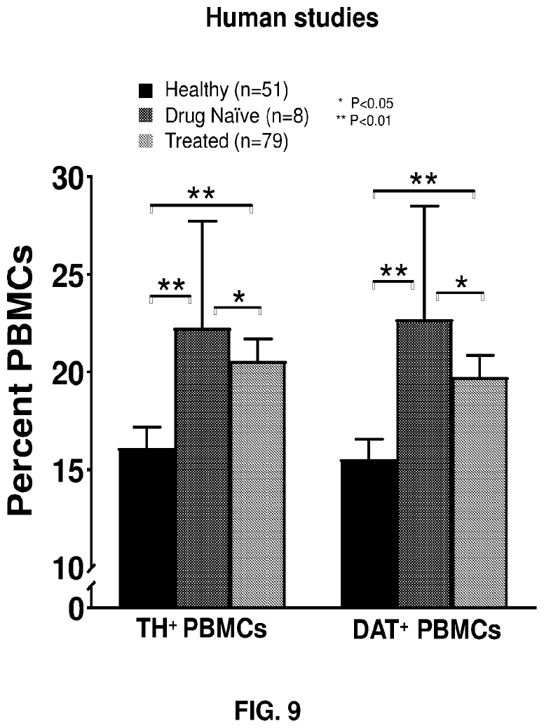

FIG. 9 is an example according to various embodiments, illustrating monocytes identified by side scatter were analyzed for TH/DAT expression as percentage of total PBMCs; alpha=0.05, one-way ANOVA with Tukey's test for multiple comparisons for whole blood acquired from PD patients and healthy, age-matched controls separated into PBMC fractions, and stained with antibodies against DAT and TH followed by appropriate secondary.

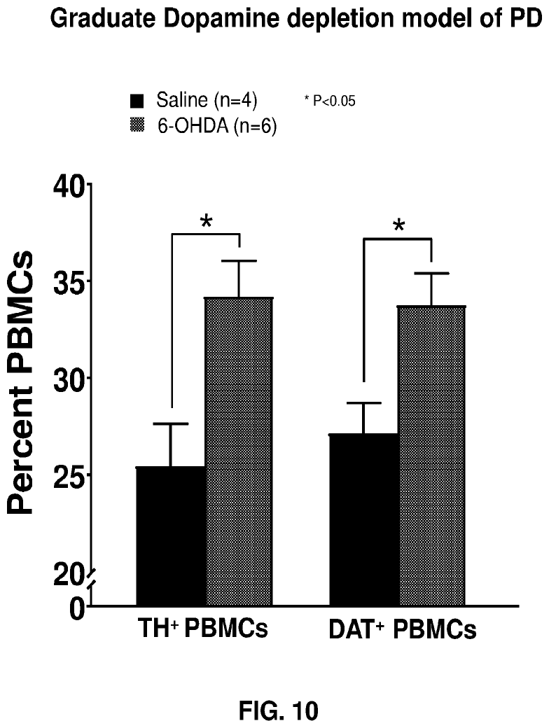

FIG. 10 is an example according to various embodiments, illustrating results for mouse monocytes isolated from gradual depletion mice treated with 6-OHDA or saline (control group) via intracranial cannula targeted to the median-forebrain bundle, and assayed for expression of DAT and TH via flow cytometry. Similar to the data in PD patients there were significant increases in (B) DAT and TH expressing monocytes in gradually depleted mice compared to saline treated control mice. Alpha=0.05, t-test with Welch's correction.

In Vitro Diagnostic Device

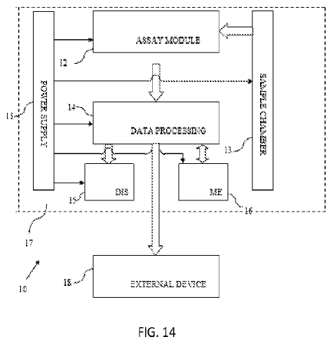

FIG. 14 schematically illustrates an inventive in vitro diagnostic device shown generally at 10. An inventive in vitro diagnostic device includes at least a sample collection chamber 13, an assay module 12 used to detect biomarkers of disease, and a user interface that relates the concentration (level) of the measured biomarker measured in the assay module. The in vitro diagnostic device may be a handheld device, a bench top device, or a point of care device.

The sample chamber 13 can be of any sample collection apparatus known in the art for holding a biological fluid. In one embodiment, the sample collection chamber can accommodate any one of the biological fluids herein contemplated, such as whole blood, cells, cell suspension, cell or tissue lysates, plasma, serum, urine, sweat or saliva.

The assay module 12 is preferably made of an assay which may be used for detecting a protein in a biological sample, for instance, through the use of antibodies in an immunoassay. The assay module 12 may include any assay currently known in the art; however, the assay should be optimized for the detection of dopamine transporter and/or tyrosine hydroxylase used for diagnosing neurological disease, severity of injury, or responsiveness to therapy in a subject. The assay module 12 is in fluid communication with the sample collection chamber 13. In one embodiment, the assay module 12 is configured to conduct an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. Alternatively, the assay module is configured to conduct a nucleic acid hybridization assay. In one embodiment a colorimetric assay may be used which may include only of a sample collection chamber 13 and an assay module 12 of the assay. Although not specifically shown these components are preferably housed in one assembly 17.

In one embodiment, the inventive in vitro diagnostic device contains a power supply 11, an assay module 12, a sample chamber 13, and a data processing module 14. The power supply 11 is electrically connected to the assay module and the data processing module 14. The assay module 12 and the data processing module 14 are in electrical communication with each other. As described above, the assay module 12 may include any assay currently known in the art; however, the assay should be optimized for the detection of the biomarkers used herein for detecting injury disease, or repair in a subject. The assay module 12 is in fluid communication with the sample collection chamber 13. The assay module 12 includes of an immunoassay where the immunoassay may be any one of a radioimmunoassay, ELISA (enzyme linked immunosorbent assay), “sandwich” immunoassay, immunoprecipitation assay, precipitin reactions, gel diffusion precipitin reactions, immunodiffusion assay, fluorescent immunoassay, chemiluminescent immunoassay, phosphorescent immunoassay, or an anodic stripping voltammetry immunoassay. A biological sample is placed in the sample chamber 13 and assayed by the assay module 12 detecting for a dopamine transmission mediator. The measured amount of the dopamine transmission mediator by the assay module 12 is then electrically communicated to the data processing module 14. The data processing 14 module may include any known data processing element known in the art, and may include a chip, a central processing unit (CPU), or a software package which processes the information supplied from the assay module 12.

In one embodiment, the data processing module 14 is in electrical communication with a display 15, a memory device 16, or an external device 18 or software package [such as laboratory and information management software (LIMS)]. In one embodiment, the data processing module 14 is used to process the data into a user defined usable format. This format includes the measured concentration (levels) of dopamine transporter and/or tyrosine hydroxylase detected in the sample, and that are useful for diagnosing neurological disease (such as Parkinson's disease), severity of injury, or responsiveness to therapy in a subject. The information from the data processing module 14 may be illustrated on the display 15, saved in machine readable format to a memory device, or electrically communicated to an external device 18 for additional processing or display. Although not specifically shown these components are preferably housed in one assembly 17. In one embodiment, the data processing module 14 may be programmed to compare the detected amount of the biomarker transmitted from the assay module 12, to a comparator algorithm. The comparator algorithm may compare the measured amount to the user defined threshold which may be any limit useful by the user. In one embodiment, the user defined threshold is set to the amount of the biomarker measured in control subject, or a statistically significant average of a control population.

In one embodiment, an in vitro diagnostic device may include one or more devices, tools, and equipment configured to hold or collect a biological sample from an individual. In one embodiment of an in vitro diagnostic device, tools to collect a biological sample may include one or more of a needle, swab, a scalpel, a syringe, a scraper, a container, and other devices and reagents designed to facilitate the collection, storage, and transport of a biological sample. In one embodiment, an in vitro diagnostic test may include reagents or solutions for collecting, stabilizing, storing, and processing a biological sample. These reagents include antibodies, aptamers, or combinations thereof raised against one of the aforementioned biomarkers. In one embodiment, an in vitro diagnostic device, as disclosed herein, may include a micro array apparatus and reagents, and additional hardware and software necessary to assay a sample to detect and visualize the temporally relevant biomarkers.

Kits

In yet another aspect, disclosed are kits for aiding a diagnosis of injury, disease, or repair, including type, phase amplitude (severity), subcellular localization, wherein the kits may be used to detect the markers of the present invention. For example, the kits can be used to detect any one or more of the biomarkers described herein, which markers are differentially present in samples of a patient and normal subjects. In another example, the kits can be used to identify compounds that modulate expression of one or more of the markers in in vitro or in vivo animal models to determine the effects of treatment.

In one embodiment, a kit includes (a) an antibody, aptamer, or nucleic acid probe that specifically binds to an aforementioned marker; and (b) a detection reagent. Such kits are prepared from the materials described above, and the previous discussion regarding the materials (e.g., antibodies, aptamers detection reagents, immobilized supports, etc.) being fully applicable to this section and thus is not repeated.

In one inventive embodiment, the kit includes (a) a composition of detecting agent to detect dopamine transporter and/or tyrosine hydroxylase.

In one embodiment, the invention includes a diagnostic kit for use in screening cells samples for dopamine transporter and/or tyrosine hydroxylase. The diagnostic kit in this embodiment includes a substantially isolated antibody or aptamer specifically immunoreactive with peptide or polynucleotide antigens, or nucleic acid probes that hybridize with polynucleotide biomarkers, and visually detectable labels associated with the binding of the polynucleotide or peptide antigen to the antibody or aptamer or nucleic acid probe. In one embodiment, the antibody or aptamer is attached to a solid support. Antibodies or aptamers used in the inventive kit are those raised against any one of the biomarkers used herein for temporal data. In one embodiment, the antibody is a monoclonal or polyclonal antibody or aptamer raised against the rat, rabbit or human forms of the biomarker. The detection reagent of the kit includes a second, labeled monoclonal or polyclonal antibody or aptamer. Alternatively, or in addition thereto, the detection reagent includes a labeled, competing antigen.

In one diagnostic configuration, test serum is reacted with a solid phase reagent having a surface-bound antigen obtained by the methods of the present invention. After binding with specific antigen antibody or aptamer to the reagent and removing unbound serum components by washing, the reagent is reacted with reporter-labeled anti-human antibody or aptamer to bind reporter to the reagent in proportion to the amount of bound anti-antigen antibody or aptamer on the solid support. The reagent is again washed to remove unbound labeled antibody or aptamer, and the amount of reporter associated with the reagent is determined. Typically, the reporter is an enzyme which is detected by incubating the solid phase in the presence of a suitable fluorometric, luminescent or colorimetric substrate.

The solid surface reagent in the above assay is prepared by known techniques for attaching protein or oligonucleotide material to solid support material, such as polymeric beads, dip sticks, a well (96-well plate) or filter material. These attachment methods generally include non-specific adsorption of the protein oligonucleotide to the support or covalent attachment of the protein or oligonucleotide, typically through a free amine group, to a chemically reactive group on the solid support, such as an activated carboxyl, hydroxyl, or aldehyde group. Alternatively, streptavidin coated plates can be used in conjunction with biotinylated antigen(s).

In some embodiments, the kit may include a standard or control information so that the test sample can be compared with the control information standard to determine if the test amount of a marker detected in a sample is a diagnostic amount consistent with a diagnosis of injury, disease, or repair, including type, phase, amplitude (severity), subcellular localization, neurodegenerative disease and/or effect of treatment on the patient.

In one embodiment, a kit includes: (a) a substrate including an adsorbent thereon, wherein the adsorbent is suitable for binding a marker (e.g. TH), and (b) instructions to detect dopamine transporter and/or tyrosine hydroxylase by contacting a sample with the adsorbent and detecting the dopamine transporter and/or tyrosine hydroxylase retained by the adsorbent. In some embodiments, the kit may include an eluant (as an alternative or in combination with instructions) or instructions for making an eluant, wherein the combination of the adsorbent and the eluant allows detection of the markers using gas phase ion spectrometry. Such kits can be prepared from the materials described above, and the previous discussion of these materials (e.g., probe substrates, adsorbents, washing solutions, etc.) is fully applicable to this section and will not be repeated.

In certain embodiments, the kit further includes instructions for suitable operational parameters in the form of a label or a separate insert. For example, the kit may have standard instructions informing a consumer how to wash the probe after a sample is contacted on the probe. In another example, the kit may have instructions for pre-fractionating a sample to reduce complexity of proteins in the sample. In another example, the kit may have instructions for automating the fractionation or other processes.

Biosamples

The inventive method and in vitro diagnostic devices provide the ability to detect and monitor levels of dopamine transporter and/or tyrosine hydroxylase which are present on or in certain cells provide enhanced diagnostic capability by allowing clinicians to determine the presence, phase and amplitude (severity) of disease. A biological sample operative herein includes cells, tissues, cell or tissue lysates, whole blood, or other biological samples recognized in the art.

Baseline levels of biomarkers (e.g. DAT or TH) are those levels obtained in the target biological sample in the species of desired subject in the absence of a known injury, disease, or repair. These levels need not be expressed in hard concentrations but may instead be known from parallel control experiments and expressed in terms of fluorescent units, density units, and the like. Typically, baselines are determined from subjects where there is an absence of a biomarker or present in biological samples at a negligible amount. However, some proteins may be expressed less in an injured, diseased or repaired patient or before any clinical measures of injury, disease, or repair. Determining the baseline levels of protein biomarkers in a particular species is well within the skill of the art.

To provide correlations between an injury, disease, or repair and measured quantities of the dopamine transporter and/or tyrosine hydroxylase, biological samples are collected from subjects in need of measurement for these biomarkers to assess injury, disease, or repair.

The detection methods may be implemented into assays or into kits for performing assays. These kits or assays may alternatively be packaged into a cartridge to be used with an inventive in vitro diagnostic device. Such a device makes use of these cartridges, kits, or assay in an assay module 12, which may be one of many types of assays. The biomarkers of the invention can be detected in a sample by a variety of conventional methods. For example, immunoassays, include but are not limited to competitive and non-competitive assay systems using techniques such as western blots, radioimmunoassay, ELISA (enzyme linked immunosorbent assay), “sandwich” immunoassays, magnetic immunoassays, radioisotope immunoassay, fluorescent immunoassays, immunoprecipitation assays, precipitin reactions, gel diffusion precipitin reactions, immunodiffusion assays, fluorescent immunoassays, chemiluminescent immunoassays, phosphorescent immunoassays, anodic stripping voltammetry immunoassay, and the like. Inventive in vitro diagnostic devices may also include any known devices currently available that utilize ion-selective electrode potentiometry, microfluids technology, fluorescence or chemiluminescence, or reflection technology that optically interprets color changes on a protein test strip. Such assays are routine and well known in the art (see, e.g., Ausubel et al., eds, 1994, Current Protocols in Molecular Biology, Vol. 1, John Wiley & Sons, Inc., New York, which is incorporated by reference herein in its entirety). Exemplary immunoassays are described briefly below (but are not intended by way of limitation). It should be appreciated, that at present, none of the existing technologies present a method of detecting or measuring any of the ailments disclosed herein, nor does there exist any methods of using such in vitro diagnostic devices to detect any of the disclosed biomarkers to detect their associated injuries.

An exemplary process for detecting the presence or absence of a biomarker, alone or in combination, in a biological sample involves obtaining a biological sample from a subject, such as a human, contacting the biological sample with a compound or an agent capable of detecting of the marker being analyzed, illustratively including an antibody or aptamer, and analyzing binding of the compound or agent to the sample after washing. Those samples having specifically bound compound or agent express the marker being analyzed.

For example, in vitro techniques for detection of a marker illustratively include enzyme linked immunosorbent assays (ELISAs), radioimmunoassay, radioassay, western blot, Southern blot, northern blot, immunoprecipitation, immunofluorescence, mass spectrometry, RT-PCR, PCR, liquid chromatography, high performance liquid chromatography, enzyme activity assay, cellular assay, positron emission tomography, mass spectroscopy, combinations thereof, or other technique known in the art. Furthermore, in vivo techniques for detection of a marker include introducing a labeled agent that specifically binds the marker into a biological sample or test subject. For example, the agent can be labeled with a radioactive marker whose presence and location in a biological sample or test subject can be detected by standard imaging techniques. It is appreciated that a bound agent assay is readily formed with the agents bound with spatial overlap, with detection occurring through discernibly different detection of each of dopamine transporter and/or tyrosine hydroxylase. A color intensity-based quantification of each of the spatially overlapping bound biomarkers is representative of such techniques.

A preferred agent for detecting a dopamine transporter and/or tyrosine hydroxylase in a biosample is an antibody, aptamer or nucleic acid probe sequence capable of binding to the biomarker being analyzed. More preferably, the antibody, aptamer or nucleic acid probe sequence is conjugated with a detectable label. Such antibodies can be polyclonal or monoclonal. An intact antibody, a fragment thereof (e.g., Fab or F(ab′)₂), or an engineered variant thereof (e.g., sFv) or an aptamer or bi-/tri-specific aptamer can also be used. Such antibodies can be of any immunoglobulin class including IgG, IgM, IgE, IgA, IgD and any subclass thereof. Antibodies and aptamers for numerous inventive biomarkers are available from vendors known to one of skill in the art. Exemplary antibodies operative herein are used to detect a biomarker of the disclosed conditions. In addition, antigens to detect autoantibodies may also be used to detect late injury of the stated injuries and disorders.

An antibody or aptamer is labeled in some inventive embodiments. A person of ordinary skill in the art recognizes numerous labels operable herein. Labels illustratively include, fluorescent labels, biotin, peroxidase, radionucleotides, or other label known in the art. Alternatively, a detection species of another antibody or aptamer or other compound known to the art is used as form detection of a biomarker bound by an antibody or aptamer.

Antibody- and aptamer-based assays operative herein include western blotting immunosorbent assays (e.g., ELISA and RIA) and immunoprecipitation assays. As one example, the biological sample or a portion thereof is immobilized on a substrate, such as a membrane made of nitrocellulose or PVDF; or a rigid substrate made of polystyrene or other plastic polymer such as a microtiter plate, and the substrate is contacted with an antibody or aptamer that specifically binds a dopamine transporter and/or tyrosine hydroxylase under conditions that allow binding of antibody or aptamer to the biomarker being analyzed. After washing, the presence of the antibody or aptamer on the substrate indicates that the sample contained the marker being assessed. If the antibody or aptamer is directly conjugated with a detectable label, such as an enzyme, fluorophore, or radioisotope, the presence of the label is optionally detected by examining the substrate for the detectable label. Alternatively, a detectably labeled secondary antibody or aptamer that binds the marker-specific antibody or aptamer is added to the substrate. The presence of detectable label on the substrate after washing indicates that the sample contained the biomarker.

Numerous permutations of these basic immunoassays are also operative in the invention. These include the biomarker-specific antibody or aptamer, as opposed to the sample being immobilized on a substrate, and the substrate is contacted with a biomarker conjugated with a detectable label under conditions that cause binding of antibody or aptamer to the labeled marker. The substrate is then contacted with a sample under conditions that allow binding of the marker being analyzed to the antibody or aptamer. A reduction in the amount of detectable label on the substrate after washing indicates that the sample contained the marker.

Although antibodies or aptamers are preferred for use in the invention because of their extensive characterization, any other suitable agent (e.g., a peptide or a small organic molecule) that specifically binds a biomarker is operative herein in place of the antibody or aptamer in the above described immunoassays. Methods for making aptamers with a particular binding specificity are known as detailed in U.S. Pat. Nos. 5,475,096; 5,670,637; 5,696,249; 5,270,163; 5,707,796; 5,595,877; 5,660,985; 5,567,588; 5,683,867; 5,637,459; and 6,011,020.

A myriad of detectable labels that are operative in a diagnostic assay for biomarker expression are known in the art. Agents used in methods for detecting a biomarker are conjugated to a detectable label, e.g., an enzyme such as horseradish peroxidase. Agents labeled with horseradish peroxidase may be detected by adding an appropriate substrate that produces a color change in the presence of horseradish peroxidase. Several other detectable labels that may be used are known. Common examples of these detectable labels include alkaline phosphatase, horseradish peroxidase, fluorescent compounds, luminescent compounds, colloidal gold, magnetic particles, biotin, radioisotopes, and other enzymes. It is appreciated that a primary/secondary antibody or aptamer system is optionally used to detect one or more biomarkers. A primary antibody or aptamer that specifically recognizes one or more biomarkers is exposed to a biological sample that may contain the biomarker of interest. A secondary antibody or aptamer with an appropriate label that recognizes the species or isotype of the primary antibody or aptamer is then contacted with the sample such that specific detection of the one or more biomarkers in the sample is achieved.

The present invention provides a step of comparing the quantity of dopamine transporter and/or tyrosine hydroxylase to normal levels to determine the disease or disorder of the subject. The results of such a test using an in vitro diagnostic device can help a physician determine whether the administration of a particular therapeutic or treatment regimen may be effective and provide a rapid clinical intervention to the injury or disorder to enhance a patient's recovery.

It is appreciated that other reagents such as assay grade water, buffering agents, membranes, assay plates, secondary antibodies or aptamers, salts, and other ancillary reagents are available from vendors known to those of skill in the art.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

### EXAMPLES

### Example 1

### Introduction

The following examples are put forth to provide those of ordinary skill in the art with a complete disclosure and description of how to perform the methods, how to make, and how to use the compositions and compounds disclosed and claimed herein. Efforts have been made to ensure accuracy with respect to numbers (e.g., amounts, temperature, etc.), but some errors and deviations should be accounted for.

The purpose of the following examples is not to limit the scope of the various embodiments, but merely to provide examples illustrating specific embodiments.

1. Materials and Methods

1.1 Materials

Whole blood was collected in K2EDTA vacutainer blood collection tubes (BD, cat. 366643) and held at room temperature for up to 2 hours prior to PBMC isolation. Primary antibodies were used as listed in Table 2. In brief, primary antibodies MAB369 and AB152 (Table 2) were used after fixation and permeabilization to detect DAT and TH respectively. Fluorochrome-conjugated anti-CD14 (MP19, BD) was used to detect CD14. Whole blood from healthy volunteers was overlaid in Leucosep tubes (Table 3) for PBMC isolation; standard 15 mL polypropylene conical tubes were used for isolation from enriched leucocytes. Specific centrifuges used are listed in Table 1.

TABLE 1
Equipment

| Equipment | Manufacturer | Model | Utility |
|---|---|---|---|
| Microcentrifuge | Fisher | 59A | FC staining |
| Centrifuge | Sorvall | ST8 | PBMC prep |

TABLE 2
Antibodies

| | | | | | Optimal | |
|---|---|---|---|---|---|---|
| Specificity | Clone | Fluorochrome | Vendor | Purpose | Titer | |
| DAT | MAB369 | unconjugated | Millipore | DA | 1 | uL |
| | | | | marker | | |
| TH | AB152 | unconjugated | Millipore | DA | 1 | uL |
| | | | | marker | | |
| CD14 | M-p19 | FITC | BD | Mono. | 2 | uL |
| | | | | Sub. | | |
| anti-Rat | Polyclonal | APC | BD | Secondary | 2.5 | uL |
| anti-Rabbit | Polyclonal | BV421 | BD | Secondary | 2.5 | uL |
| CD16 | 3G8 | APC | Biolegend | Mono. | 2 uL (mfg) | |
| | | | | Sub. | | |
| Mouse | IgG2b, k | FITC | Biolegend | Isotype | 2 | uL |
| Isotype | | | | | | |
| control | | | | | | |
| Rat Isotype | RTK2758 | unconjugated | Biolegend | Utility | 0.1 | uL |
| control | | | | | | |
| Rabbit | Polyclonal | unconjugated | Biolegend | Utility | 0.1 | uL |
| Isotype | | | | | | |
| control | | | | | | |

TABLE 3
Reagents

| | | Catalog |
|---|---|---|
| Name | Vendor | Number |
| NearIR Fixable Viability Dye | ThermoFisher | L34975 |
| Fetal Bovine Serum | Gemini | 100-106 |
| DMSO | Sigma | D2438-10ML |
| RPMI 1640 (−) Phenol Red | Gibco | 11835030 |
| Fix/Perm Kit | eBioscience | 88-8824-00 |
| Trypan Blue | MP Biomedicals | 1691049 |
| Ficoll-Paque PLUS | GE Healthcare | 17144003 |
| CryoStor10 | Sigma | C2874 |

1.2 Human Samples

Human blood samples were purchased from Lifesouth Community Blood Center, Gainesville, Fla. from April, 2017 to April, 2018. The study was approved by the University of Florida's Institutional Review Board (IRB). According to Lifesouth rules and regulations donors were healthy individuals aged 20-70 years-old of any gender; who were not known to have any blood borne pathogens, and were never diagnosed with a blood disease, such as leukemia or bleeding disorders. In addition, none of the donors were using any medications for an infection, nor were they on any blood.

1.3 Flow Cytometer

Beckton Dickenson Canto II 3 laser system with a (50 MW 488 nm blue sapphire laser, 100 MW 405 nm violet laser and a 50 MW 633 Red laser) with 8 fluorescent detectors, 2 off the violet laser, 4 off of the blue laser and 2 off the red laser (405 NM filters; 450/50, 510/50; 488 NM filters 530/30, 585/42, 710/20, and 780/60; 633 NM filters; 660/20 and 780/60) and FSC/SSC for a total of 10 parameters. Daily QC is preformed using BD FACSDiva CS&T research beads; Cat. 655051) and by running twice weekly Spherotech 8 peak rainbow Calibration beads (Cat. RCP-30-5A (8 peaks). All QC records are keep in digital format on the instrument and available for ICBR Flow Core users to examine.

The acquisition of each sample was stopped when total events reached 100,000. Each sample data set consisted of: cell morphology and size data by sidescatter (SSC) and forwardscatter (FSC) respectively, and fluorescence data for each event in channels detecting FITC, APC, Pacific Blue/Brilliant Violet 421, and APC-Cy7/NearlR, to accurately detect each fluorochrome labeled antibody against proteins of interest (Table 1).

1.4 Cell Sample Preparation

1.4.1 Pbmc Isolation:

Briefly, enriched leucocytes/whole blood was diluted 1:1 in PBS and layered atop Ficoll and centrifuged to generate PBMC layer. PBMCs were aspirated, washed twice with sterile PBS and prepared for staining.

The volume of whole blood and enriched leucocytes, dilute and layered atop Ficoll, varied depending on condition of the sample—as whole blood contains a greater percentage of red blood cells (RBCs) in contrast to enriched leucocytes, whole blood required an altered ratio of dilute blood to Ficoll (2:1) compared to enriched leucocytes (3.3:1) to compensate for increased percentage RBCs in the sample. 30 mL dilute whole blood was layered atop 15 mL Ficoll in a sterile 50 mL Leucosep Tube (Table 3), while 10 mL dilute enriched leucocytes was layered atop 3 mL Ficoll in a 15 mL conical tube.

1.4.2 Pbmc Cryopreservation:

FBS-DMSO

To compare the results of freshly isolated PBMCs to a commonly used cryopreservation method, 10 million isolated PBMCs were suspended in 900 uL FBS (Table 3) and 100 uL sterile DMSO added dropwise to a final volume of 1 mL. Suspension was quickly transferred to a cryopreservation tube and into a cryopreservation canister pre-chilled to −20 C. The canister was stored at −80 C for 48 hours before moving samples into liquid nitrogen for a minimum of 24 hours. The cryopreserved PBMCs thawed at 37 degrees C. for 2 minutes until a small ice crystal remained. PBMCs were suspended in 9 volumes of RPMI 1640 without phenol red, containing 10% FBS, centrifuged at 100 g for 10 min, and washed with 2 volumes of sterile PBS.

CryoStor10

To compare the results of freshly isolated PBMCs to specialized cryopreservation methods, 10 million isolated PBMCs were suspended in 100 uL PBS and added to a cryotube. Sterile CS10 freezing media (Table 3) at 4 degrees C. was added dropwise and agitated gently by hand every 5 drops for a total volume of 1 mL. Cryopreservation tube was quickly transferred to a cryopreservation canister pre-chilled to −20 C. The canister was stored at −80 C for 48 hours before moving samples into liquid nitrogen for a minimum of 24 hours. The cryopreserved PBMCs quick-thawed at 37 degrees C. for 2 minutes until a small ice crystal remained. PBMCs were suspended in 9 volumes of sterile PBS, centrifuged at 100 g for 10 min, and washed with 2 volumes of sterile PBS to remove residual freezing media.

1.5 Staining Conditions and Compensation:

Staining was performed in serum-free PBS. Cryopreserved PBMC samples included incubation with NearlR fixable viability dye (Table 3) for 15 minutes at room temperature in PBS, followed by quenching with PBS containing 1% FBS (Table 3). Fc-blocking (anti-CD16/32) is often used to avoid nonspecific antibody binding in immune cells—no altered fluorescent signals were found in presence or absence of Fc-block. Every sample was stained in parallel with unstained, secondary only and isotype controls. Staining was conducted for 30 minutes on ice (live cells) and 30 minutes at room temperature (after fixation) protected from light. After testing fixation and permeabilization with 4% PFA followed by a series of Triton-X100 concentrations from 0.001% to 0.5%, it was found that cell morphology and staining efficiency was negatively impacted. Therefore, according to various embodiments an eBioscience fixation and permeabilization kit may be used (Table 3) to fix live cells for 45 minutes at room temperature, and washed following manufacturer recommendations, to produce reliable intracellular staining.

Each set of experiments was compensated using single color compensation controls of stained PBMCs using each fluorochrome-conjugated antibody listed in Table 2 Viability dye compensation was achieved by using 1:1 live cells combined with cells heat-killed at 56 C for 5 min.

1.5.1 Staining Procedure—Freshly Isolated and Cryopreserved PBMCs:

Cell pellet was resuspended in 100-400 uL sterile PBS depending on qualitative pellet size, counted and density adjusted to 10,000 cells per microliter, and aliquoted 1 million cells per staining condition. Samples were incubated with 2 uL anti-CD14 (BD, M-p19) (Table 2) for 30 minutes on ice, washed twice with ice cold PBS, and suspended in 100 uL fixation buffer for 45 minutes. Following two washes with permeabilization buffer, samples were stained with anti-DAT (1 uL, Sigma; Cat. MAB369) and anti-TH (1 uL, Sigma; Cat. AB152) as well as isotype controls (Table 2), followed by 2.5 uL of species matched secondary antibodies (Table 2) for 30 minutes. Samples were washed twice to remove unbound antibody and suspended in 500 uL PBS; data was acquired within 2 hours of staining.

1.5.2 Staining Procedure—Step-by-Step

Refer to Table 1 (Equipment), 2 (Antibodies) and 3 (Reagents)

Suspend cell pellet in 100-400 uL sterile PBS depending on pellet size. Dilute 1:200 in PBS containing 10% Trypan Blue, count, excluding dead cells and density adjust to 10,000 cells/uL. Aliquot 1 million cells into each of 4 staining tubes: Unstained, Secondary Only, Isotype control, and Stained. Repeat for each independent biological sample. Use one set of sample tubes for single color compensation controls.

Incubate on ice: stained sample with anti-CD14-FITC 1:50 dilution, Isotype control with mouse-IgG2b,K-FITC 1:50 dilution. Incubate for 30 minutes on ice protected from light.

Wash with ice-cold PBS—centrifuge at 4 C for 5 minutes at 1800 RPM in a swinging bucket microcentrifuge (Table 1), aspirate supernatant, resuspend and repeat for a total of two washes.

For cryopreserved PBMCs—add NearIR fixable viability dye at 1:1000 final dilution, incubate at room temperature for 15 minutes protected from light. Quench unbound dye with 900 uL PBS containing 1% FBS, immediately centrifuge and aspirate supernatant as above. Proceed to fixation.

Fix and permeabilize samples following kit manufacturer instructions (Table 3) except using 1 mL permeabilization buffer instead of 2 mL per wash step. Resuspend all samples in final volume 100 uL permeabilization buffer.

Add primary antibodies against DAT and TH (Table 2) 1:100 dilution to appropriate tubes, and 1:1000 dilution of each isotype control to appropriate tubes.

Incubate 30 minutes at room temperature protected from light. Wash as above with permeabilization buffer.

Add secondary antibodies (Table 2) at 1:40 dilution to appropriate tubes, and incubate as in step 5. Follow with wash steps.

Resuspend final pellet in 500 uL sterile PBS. Acquire data on instrument within 2 hours of staining completion.

1.5.3 Comparing PBMCs Freshly Isolated to Cryopreserved Obtained from the Same Donor:

Immediately following PBMC isolation from whole blood or enriched leucocytes, 10 million cells were cryopreserved (2.4.2) while an equivalent number of PBMCs were used for immediate analysis. Cryopreserved PBMCs from the same isolation were thawed and stained as above. Direct comparisons were made following post-hoc analysis between freshly isolated and cryopreserved PBMCs from each independent biological replicate. To examine effects of cryopreservation on monocyte subsets, 2 uL of an antibody against CD16 (Biolegend, cat. 302011) (2, 12) was added along with anti-CD14 in a separate staining tube. Post-hoc analysis in FCSExpress6 RUO followed by statistical analysis with computer software for analyzing and graphing scientific data, specifically GRAPHPAD PRISM® 7 software, was used to determine the similarities and differences in monocyte subsets in freshly isolated versus cryopreserved PBMCs from the same donor.

1.5.4 Data Acquisition:

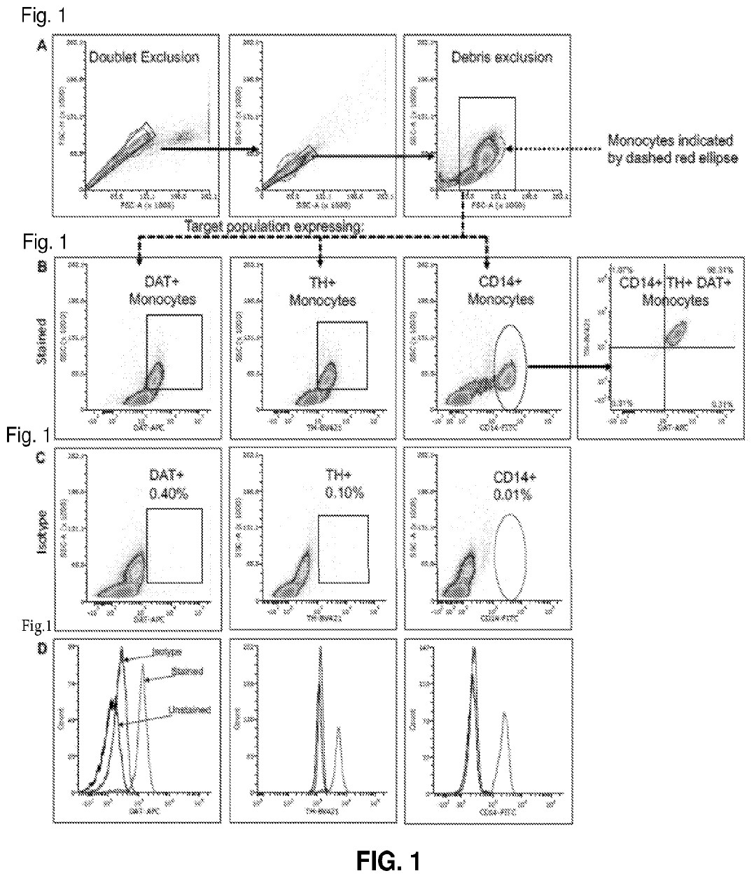

All fluorochromes were compensated using BD FACSDiva automatic compensation calculation. In brief, 5,000 events of each single-color compensation control were acquired in BD FACSDiva, monocytes were gated by light scatter (shown in FIG. 1b, left), and compensation gates were set on histogram plots for each fluorochrome. Spectral overlap was calculated and automatically applied to all samples.

Results

Step 1: PBMC Preparation

Multiple lines of evidence suggest a role for blood leukocytes in dopamine transmission and thus the goal of this work was to characterize expression of key dopaminergic proteins such as dopamine transporter (DAT) and tyrosine hydroxylase (TH). Leukocytes exist as suspended cells in blood, and thus flow cytometry is a suitable method to study these dopaminergic proteins in peripheral blood-borne immune cells.

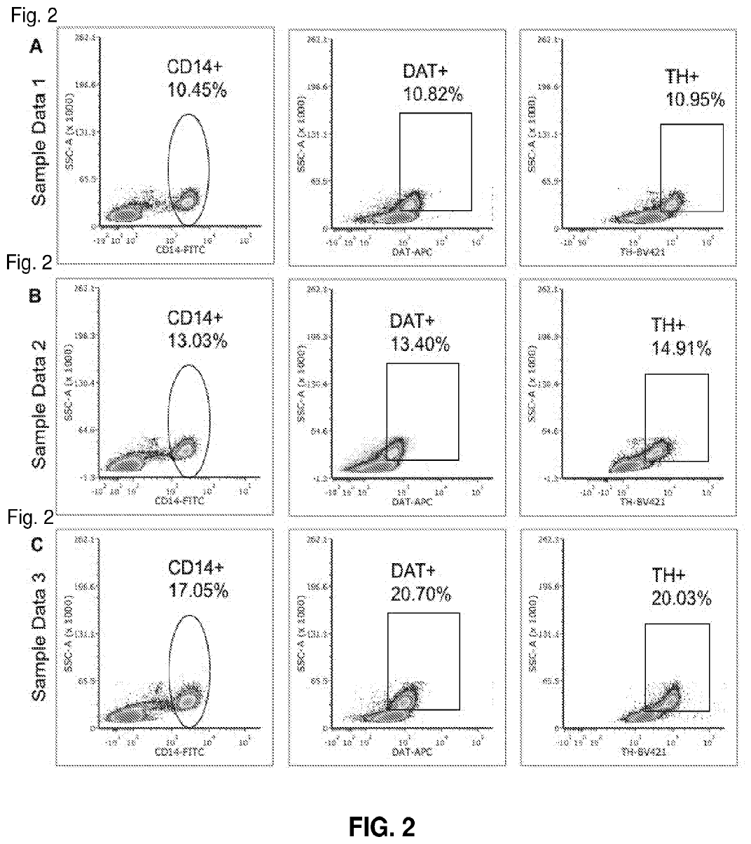

The reliability of Ficoll density centrifugation for isolation of viable PBMCs from whole blood is well established (6), and provides the added benefit of excluding granulocytes from analysis which may confound analysis of monocyte populations. While flow cytometry is routinely used for detection and analysis of immune cell such as monocytes, it has not been used to detect TH and DAT in the human PBMCs; results obtained according to various embodiments have shown that human PBMCs express these two markers of dopamine systems both at message and protein level (10). Various embodiments provide an optimized protocol to detect DAT and TH in monocytes by flow cytometry, in freshly isolated human PBMCs as well as in the cryopreserved PBMCs. The accuracy of DAT and TH detection was assessed by right-ward shift in the fluorescence intensity relative to negative controls; percentage monocytes expressing DAT and TH were calculated as a percentage of total PBMCs. Both freshly isolated and cryopreserved (see sections 3.6 and 3.7) samples yielded viable PBMCs, as determined by trypan blue staining. Low cellular debris was detected in both freshly prepared and cryopreserved samples (fresh PBMC isolation shown in FIG. 1A). The density dot plot obtained from FSC×SSC visualization revealed two distinct cell populations in both cryopreserved and freshly prepared samples (FIG. 1A, FIG. 2).

FIGS. 1A, 1B, 1C, and 1D are examples according to various embodiments, generally illustrating that flow cytometry reliably detects DAT and TH expression in human PBMCs obtained from whole blood. More specifically, FIG. 1A is an example according to various embodiments illustrating representative plots showing the gating strategy_for DAT-expressing and TH-expressing human monocytes by flow cytometry, and more specifically doublet discrimination and debris exclusion. FIG. 1B is an example according to various embodiments illustrating representative plots showing the gating strategy_for DAT- and TH-expressing human monocytes by flow cytometry, and more demonstrating that DAT and TH expressing monocytes may be discriminated based on sidescatter properties intrinsic to monocytes (B, 1^{st} and 2^{nd} panel from left) or by expression of human monocyte marker CD14 (B, 3rd and 4th panel from left). FIG. 1C is an example according to various embodiments illustrating representative plots showing the gating strategy_for DAT- and TH-expressing human monocytes by flow cytometry, and more specifically demonstrating that isotype controls reveal minimal nonspecific staining. FIG. 1D is an example according to various embodiments illustrating representative plots showing the gating strategy_for DAT- and TH-expressing human monocytes by flow cytometry, and more specifically histogram representations for anti-DAT, anti-TH and anti-CD14 displaying mean fluorescence intensity (MFI) for: unstained (black), isotype control (blue) and immune-stained (red). N=41 independent biological replicates.

FIGS. 2A, 2B, 2C, 2D, and 2E are examples according to various embodiments illustrating that PBMC cryopreservation in FBS-DMSO alters flow cytometric detection of DAT and TH. FIG. 2A is an example according to various embodiments illustrating freshly isolated PBMCs show DAT, TH and CD14 expressing monocytes comparable to baseline for healthy individuals. FIG. 2B is an example according to various embodiments illustrating cryopreserved in FBSDMSO and quick-thawed PBMCs isolated from the same donor show dramatically altered percentage of PBMCs expressing DAT, TH and CD14. FIG. 2C is an example according to various embodiments illustrating cryopreserved PBMCs in FBSDMSO show clear CD14 staining, but a significant reduction of CD16 signal. FIG. 2D is an example according to various embodiments illustrating altered percentage of DAT and TH expressing PBMCs before and after freeze-thaw. FIG. 2E is an example according to various embodiments illustrating significantly altered percentage of CD14+ to CD16+ monocytes shows the consequences of cryopreservation. Statistical analysis conducted with computer software for analyzing and graphing scientific data, specifically GRAPHPAD PRISM® software, using multiple t tests with Holm-Sidak correction for multiple comparisons; alpha=0.05. N=3 independent biological replicates.

Step 2: Gating Strategy for Single Cell Analysis of Monocytes:

As shown in FIG. 1A, doublets were excluded by overlapping nested gates—single cells were first isolated by gating events on FSC-A (area)×FSC-H (height) followed by an additional overlapping gate on SSC-A×SSC-H. Less than 1% of events were excluded in the final doublet exclusion gate, indicating that only single cells were included in subordinate gates. The target population was isolated from cellular debris by including the events with FSC values greater than 45,000. Lymphoid and myeloid cells were clearly delineated in the final gate as two distinct populations with identifying FSC and SSC properties—the lymphoid population exhibits lower values on both FSC and SSC, with monocytes exhibiting higher FSC and SSC values (shown by dashed red ellipse). While lymphoid cells do express DAT and TH to varying degrees (9, 12, 13), monocytes were the specific population of interest and studied exclusively in this analysis. Monocytes were gated from lymphocytes by sidescatter for further analysis of CD14, DAT and TH expression.

Step 3: Fixation and Permeablization for Intracellular Staining

TH is a cytosolic protein. The DAT antibody used in this study detects an intracellular N-terminus epitope (3, 5, 8, and 11); therefore, both TH and DAT staining required permeabilization step. Multiple fixations and permeabilization strategies were tested to achieve reproducible results. The optimum fixation and permeabilization strategies for DAT and TH detection is described in the Materials and Methods section. While fixation using PFA proved reliable for flow cytometry detection of membrane proteins, the Triton X-100 permeabilization at any concentration affected cell viability, morphology and staining efficiency. Therefore, in this study to detect DAT and TH expressing monocytes, an eBioscience© fixation and permeabilization kit (cat. 88-8824-00) was used (Table 3) which did not affect cell morphology or staining efficiency and produced reproducible staining in 41 independent staining of human samples.

Step 4: Optimization of DAT and TH Detection by Flow Cytometry

Antibody staining conducted in a final staining volume of 100 uL containing 1 million cells yielded consistent and reproducible results detecting both DAT and TH in monocytes, as measured by increased fluorescence intensity over non-immune isotype negative control of at least 1 log unit. Results shown in FIGS. 1, 2 and 3 represent the optimum dilution factor of 1:100 from stock. Isotype controls for each antibody were included as the main negative control at an identical final concentration and yielded negligible levels of nonspecific background binding (FIG. 1C).

DAT and TH Expression in Monocytes can be Reliably Detected by Flow Cytometry

Presence of DAT and TH in human monocytes has been demonstrated by western blot, qPCR, and immunofluorescent assays (7, 10). While these methodologies are reliable and proven, they are time consuming and unsuitable for use in clinical setting or the real-time comparison of large number of samples. Therefore, there is an unmet need to develop a standardized and high throughput methodology to rapidly and reliably detect these proteins at basic science laboratories and/or standard clinical facilities. Various embodiments provide a sensitive flow cytometry panel to detect DAT and TH in human monocytes. FIG. 1 shows clear separation of at least 1 log unit between monocytes incubated with non-immune isotype antibody (FIG. 1C) and those incubated with anti-DAT and anti-TH (FIG. 1B). While most but not all (the majority) of human monocytes express TH and DAT, it was found that ˜100% of CD14+ monocytes co-express these two proteins (FIG. 1B). Therefore, this study assayed for CD14+ monocytes co-expressing TH and DAT to ensure consistency across samples. Applying this strategy, samples that produced less than 75% TH and DAT co-expression in CD14+ monocytes suggested under-staining and were excluded from analysis.

These experiments were repeated in 41 whole blood samples drawn from healthy volunteers across different ages/genders (Table 3). Data obtained in 41 independent experiments suggest DAT and TH expressing monocytes comprise ˜12% of the PBMC fraction in the healthy human (sample data shown in FIG. 2), providing a dependable methodology to analyze the expression of these two dopaminergic markers in human blood samples in health and disease.

Cryopreservation with FBS/DMSO Alters the Immune Phenotype of Monocytes.

PBMCs are routinely cryopreserved for long-term studies, transport to research labs across the world and other practical considerations. Therefore, the applicability of the panel according to various embodiments was tested to detect DAT, TH, CD14 and CD16 in cryopreserved PBMCs.

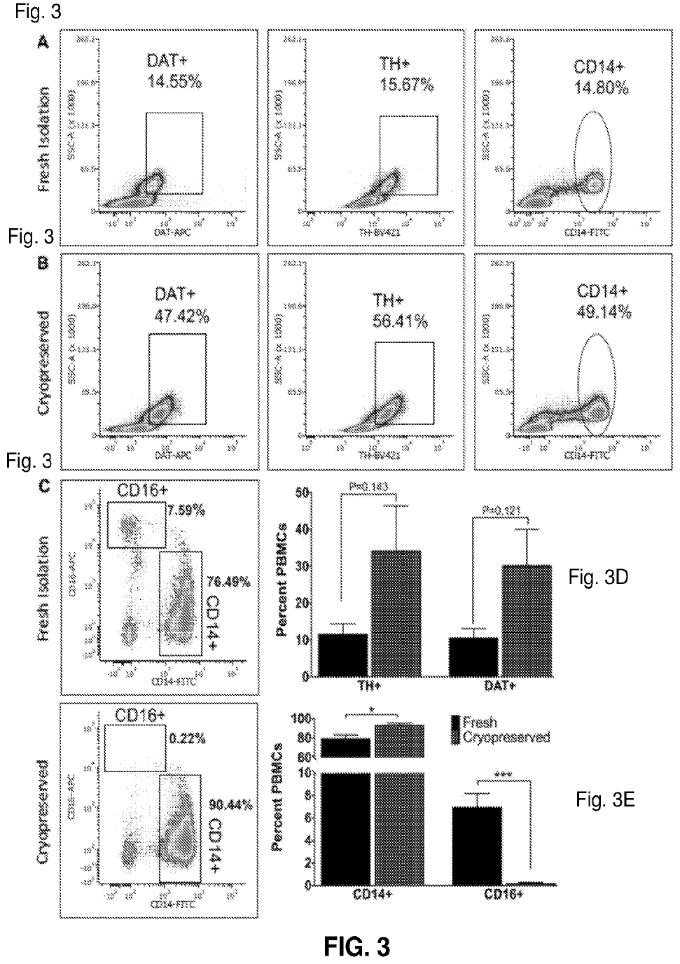

FIGS. 3A, 3B, 3C, 3D, and 3E are examples according to various embodiments illustrating that PBMC cryopreservation in FBS-DMSO alters flow cytometric detection of DAT and TH. FIG. 3A is an example according to various embodiments illustrating freshly isolated PBMCs show DAT, TH and CD14 expressing monocytes comparable to baseline for healthy individuals. FIG. 3B is an example according to various embodiments illustrating cryopreserved in FBS-DMSO and quick-thawed PBMCs isolated from the same donor show dramatically altered percentage of PBMCs expressing DAT, TH and CD14. FIG. 3C is an example according to various embodiments illustrating cryopreserved PBMCs in FBS-DMSO show clear CD14 staining, but a significant reduction of CD16 signal. FIG. 3D is an example according to various embodiments illustrating altered percentage of DAT and TH expressing PBMCs before and after freeze-thaw. FIG. 3E is an example according to various embodiments illustrating significantly altered percentage of CD14+ to CD16+ monocytes shows the consequences of cryopreservation. Statistical analysis conducted with computer software for analyzing and graphing scientific data, specifically GRAPHPAD PRISM® software, using multiple t tests with Holm-Sidak correction for multiple comparisons; alpha=0.05. N=3 independent biological replicates.

In three independent biological replicates, freshly isolated PBMCs were compared to PBMCs frozen in FBS with 10% DMSO immediately after isolation and found that in each sample the percentages of PBMCs expressing DAT and TH were altered compared to freshly isolated PBMCs (FIG. 3a, b). To determine whether this change was due to a loss in a specific subset of monocytes, monocyte subsets were assayed for expression of an additional monocyte marker. Surprisingly, it was found while freshly isolated PBMCs showed a stable presence of CD16+ PBMCs, this population is almost absent in the FBS-DMSO cryopreserved cells suggesting a loss of this phenotype during cryopreservation and thaw. Therefore, cryopreservation of PBMCs using FBS/DMSO alters the immune phenotype of monocyte subsets included in the PBMC fraction such that these are not equivalent to those in freshly obtained cells.

### Cryopreservation with CryoStor10 Maintains the Immune Phenotype of Monocytes

Since cryopreservation in FBS-DMSO alters the immune phenotype of monocytes, many other cryopreservation strategies were examined. A proprietary serum-free freezing media CryoStor10 (CS10, Sigma, Cat. C2874, Table 3), designed to manage cellular stress during freezing, was identified to determine whether freezing in serum-free media is more suitable for DAT/TH detection by flow cytometry following PBMC cryopreservation.

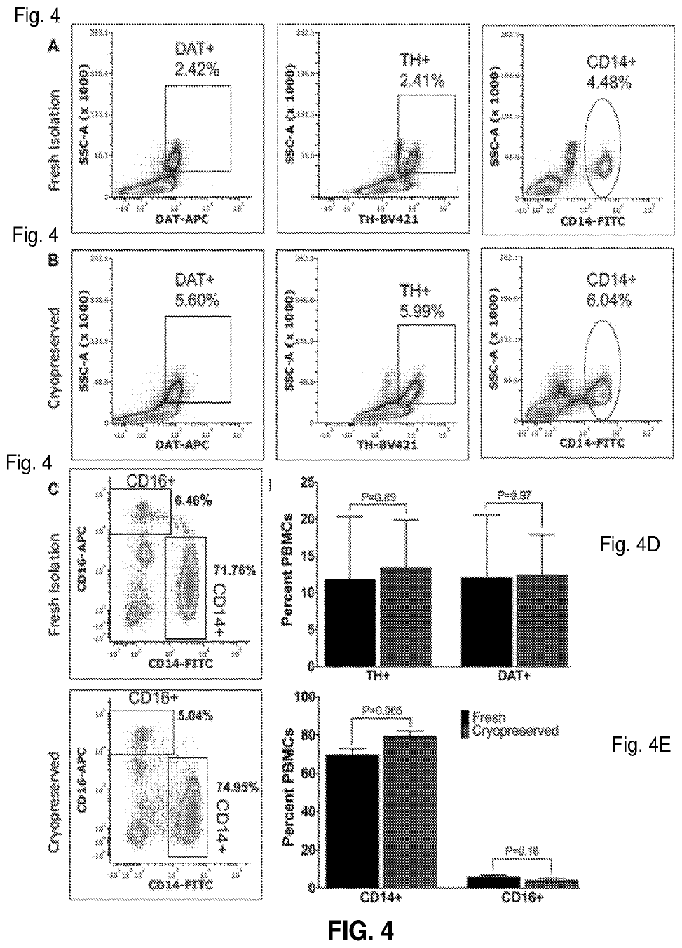

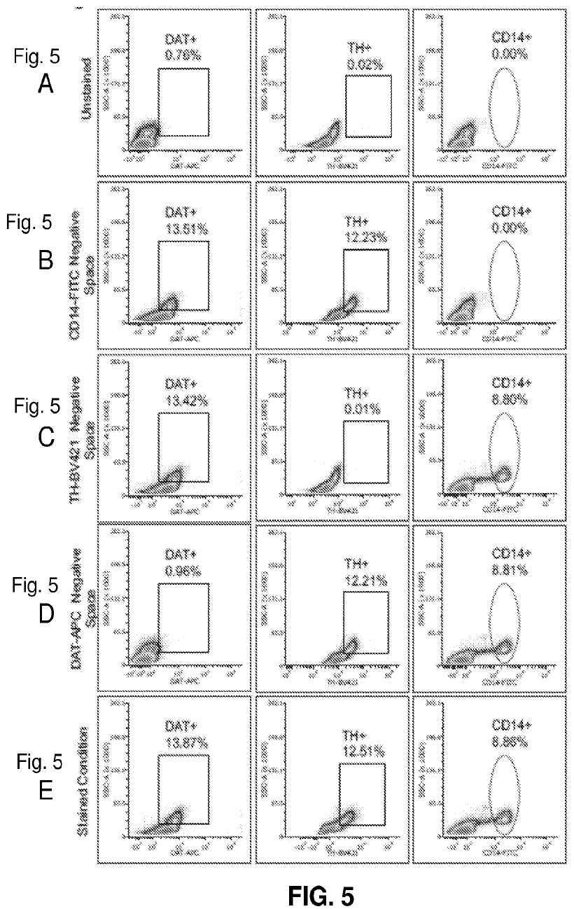

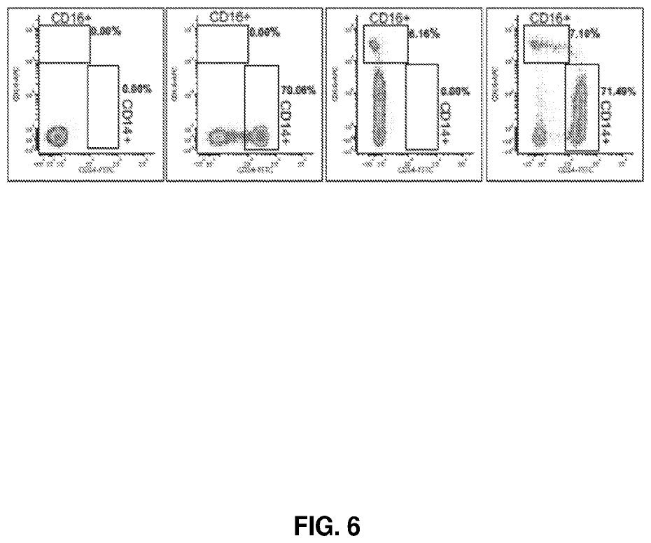

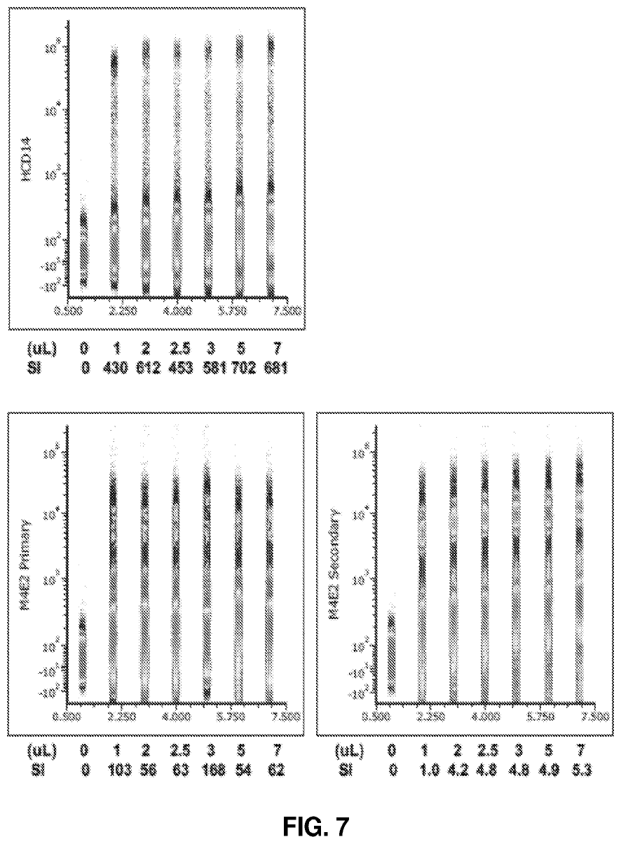

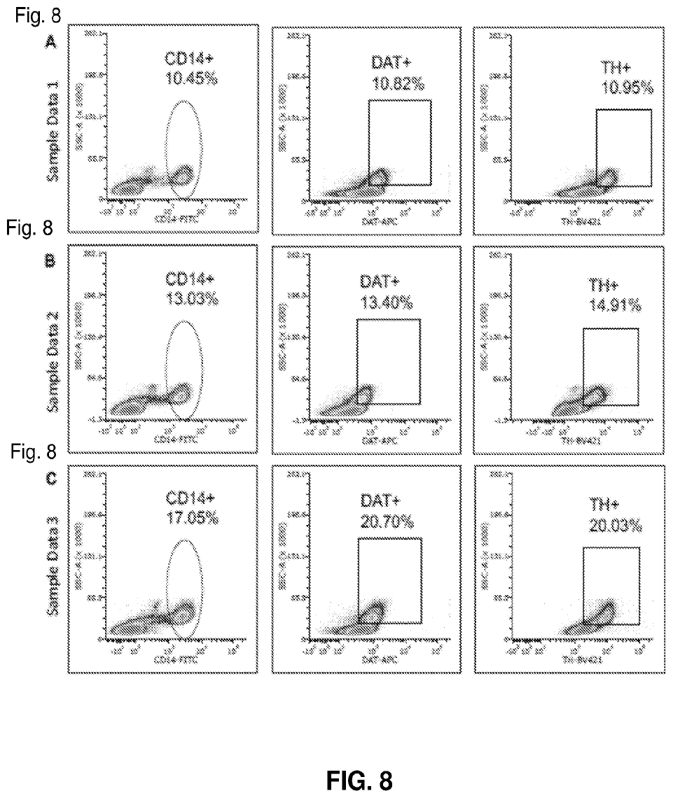

In three independent biological replicates, freshly isolated PBMCs were compared to PBMCs frozen in CS10 immediately after isolation. In each of three replicates, percentages of PBMCs expressing DAT and TH were not significantly different compared to freshly isolated PBMCs (FIG. 4a, b). The monocyte subsets were assayed with CD16 as a parallel to the earlier study of cryopreserved monocytes. While CD14 monocyte populations are largely intact, CD16+ monocytes are slightly but non-significantly affected by cryopreservation in CS10, suggesting that cryopreservation with CS10 is a viable preservation method that maintains the immune phenotype integrity of monocytes required in flow cytometry assay for detection of TH and DAT.

### DISCUSSION

While flow cytometry has been routinely used for immunological studies, this report presents the first use of flow cytometry to study the dopaminergic proteins DAT and TH in peripheral monocytes. Dysregulated dopamine homeostasis is characteristic of a variety of degenerative diseases, i.e. Parkinson's disease, and increasing evidence correlate the dysregulation of peripheral dopamine signaling with dysfunction of CNS dopaminergic function. However, the search for peripheral biomarkers of CNS disease has yielded disappointing results thus far. The flow cytometry panel according to various embodiments provides a technique to study dysregulation of peripheral dopamine homoeostasis in immune cells, potentially paving the way to biomarker studies.

Multiple repositories for human blood and PBMCs, employing various cryopreservation methods, are already available. The performance of the panel according to various embodiments was characterized in multiple cryopreservation methods and identified the most suitable method. Further, it was determined that cryopreservation using FBS/DMSO is unsuitable for this specific panel, as a marked loss of one monocyte phenotype was observed. An alternate cryopreservation method was identified using CS10 that produced markedly better results. This disparity may be due to immune activation by serum (FBS) or as consequence of additional proprietary cryoprotectants present in CS10 that enable preservation of delicate monocyte subsets for future analysis.

The instrument used in these studies, BD Canto II, is equipped to detect up to 8 appropriately selected fluorochromes; as the panel according to certain embodiments only includes three fluorochromes, a variety of additional markers of interest can be studied in parallel expanding the potential of studying dopaminergic proteins on leukocytes included in the PBMC fraction.

The primary challenge in establishing this new flow cytometry panel was to determine the appropriate conditions in which to detect DAT and TH in PBMC fraction while maintaining cell viability, maximizing signal to background ratios and to allow reliable detection of these markers in an innately heterogeneous human population. Studies applying flow cytometry in neuroscience have often relied on transgenic animal studies expressing fluorescent reporter proteins, or utilized commercially available antibodies against common immunological targets which are typically directly fluorochrome-conjugated and thus require minimal optimization. The absence of flow cytometry tested antibodies designed for neuroscience applications has greatly limited application of this technique; it was elected to establish this protocol detecting dopaminergic proteins in peripheral cells with the expectation that this will be used to inform the ongoing search for mechanistic insights and peripheral biomarkers in CNS-associated diseases involving dysregulated dopamine homeostasis.

### REFERENCES RELATED TO EXAMPLE 1

1. Autissier, P., Soulas, C., Burdo, T. H., and Williams, K. C. (2010). Evaluation of a 12-color flow cytometry panel to study lymphocyte, monocyte, and dendritic cell subsets in humans. Cytometry Part A
2. Boyette, L. B., Macedo, C., Hadi, K., Elinoff, B. D., Walters, J. T., Ramaswami, B., Chalasani, G., Taboas, J. M., Lakkis, F. G., and Metes, D. M. (2017). Phenotype, function, and differentiation potential of human monocyte subsets. PLOS ONE 12, e0176460.
3. Ciliax, B. J., Heilman, C., Demchyshyn, L. L., Pristupa, Z. B., Ince, E., Hersch, S. M., Niznik, H. B., and Levey, A. I. (1994). The Dopamine Transporter: Immunochemical characterization and localization in brain. Jneurosci 15, 1714-1723.
4. Eisenhofer, G., Åneman, A., Friberg, P., Hooper, D., Fåndriks, L., Lonroth, H., and Mezey, E. (1997). Substantial Production of Dopamine in the Human Gastrointestinal Tract. 82, 8.
5. Eriksen, J., Rasmussen, S. G. F., Rasmussen, T. N., Vaegter, C. B., Cha, J. H., Zou, M.-F., Newman, A. H., and Gether, U. (2009). Visualization of Dopamine Transporter Trafficking in Live Neurons by Use of Fluorescent Cocaine Analogs. Journal of Neuroscience 29, 6794-6808.
6. Fluks, A. J. (1981). Three-step isolation of human blood monocytes using discontinuous density gradients of percoll. Journal of Immunological Methods 41, 225-233.
7. Gaskill, P. J., Carvallo, L., Eugenin, E. A., and Berman, J. W. (2012). Characterization and function of the human macrophage dopaminergic system: implications for CNS disease and drug abuse. Journal of Neuroinflammation 9.
8. Hersch, S. M., Yi, H., Heilman, C. J., Edwards, R. H., and Levey, A. I. (1997). Subcellular localization and molecular topology of the dopamine transporter in the striatum and substantia nigra. The Journal of Comparative Neurology 388, 211-227.
9. Kustrimovic, N., Rasini, E., Legnaro, M., Bombelli, R., Aleksic, I., Blandini, F., Comi, C., Mauri, M., Minafra, B., Riboldazzi, G., et al. (2016). Dopaminergic Receptors on CD4+T Naive and Memory Lymphocytes Correlate with Motor Impairment in Patients with Parkinson's Disease. Scientific Reports 6.
10. Mackie, P., Lebowitz, J., Saadatpour, L., Nickoloff, E., Gaskill, P., and Khoshbouei, H. (2018). The dopamine transporter: An unrecognized nexus for dysfunctional peripheral immunity and signaling in Parkinson's Disease. Brain, Behavior, and Immunity 70, 21-35.
11. Miller, G. W., Staley, J. K., Heilman, C. J., Perez, J. T., Mash, D. C., Rye, D. B., and Levey, A. I. (1997). Immunochemical analysis of dopamine transporter protein in Parkinson's disease. Annals of Neurology 41, 530-539.
12. Mukherjee, R., Kanti Barman, P., Kumar Thatoi, P., Tripathy, R., Kumar Das, B., and Ravindran, B. (2015). Non-Classical monocytes display inflammatory features: Validation in Sepsis and Systemic Lupus Erythematous. Scientific Reports 5.
13. Pinoli, M., Marino, F., and Cosentino, M. (2017). Dopaminergic Regulation of Innate Immunity: a Review. Journal of Neuroimmune Pharmacology 12, 602-623.

### Example 2

### Overview

Changes in tyrosine hydroxylase (TH), the rate limiting enzymatic step in synthesis of catecholamines norepinephrine and dopamine, has been implicated in many neurological and neuropsychiatric diseases. Traditionally, TH is detected via methods such as western blot, immunohistochemistry and immunocytochemistry. These methodologies use antibodies generated against TH and only provide qualitive assessments of TH expression. There is an unmet need to develop sensitive assays for the quantitative measurement of TH level that is critical for diagnostic and assessment of therapeutic strategies. Described in this Example 2 is a highly sensitive and reproducible enzyme linked immunosorbent assay (ELISA) capable of detecting TH protein as low as 15 pg/mL of TH protein. Further, it has been demonstrated that the assay detects TH in PC12 cell lines, primary murine dopamine neurons, human monocyte-derived macrophages, and primary human monocytes isolated from peripheral blood.

Having demonstrated applicability of the novel ELISA in cells directly isolated from human blood, the potential of this tool as a device to study human disease in the clinical setting was realized. In order to test the applicability of the assay to human disease, it was investigated whether patients with Parkinson's disease, in which a reduction in TH-expressing neurons results in movement disorder, had altered expression of TH in peripheral monocytes. In 5 Parkinson's patients and 8 healthy control subjects recruited via an approved IRB protocol, it was found that the blood monocytes of patients with Parkinson's disease exhibit significantly more TH protein relative to healthy controls. Since Parkinson's disease is traditionally studied as a disease of the central nervous system in which a degeneration of TH expressing neurons results in the symptoms of Parkinson's disease, the presence of altered TH expression in peripheral blood monocytes provides an unexpected immune correlate of brain changes.

These results show that, in a disease in which brain dopamine neuron degeneration is the traditional treatment target, TH in the peripheral immune system is altered indicating a potential for communication between the brain and peripheral immune system in Parkinson's disease. Furthermore, this result suggests that the novel, highly sensitive and low-cost assay has exciting applications in diagnosis, disease monitoring and assessment of treatment efficacy in Parkinson's patients, as well as in other conditions in which monoamines such as norepinephrine and dopamine are dysregulated—including addiction, bipolar disorder and schizophrenia.

### Materials and Methods

TABLE 4
Antibodies

| Specificity | Clone/Species | Conjugate | Vendor | Catalog Number | Purpose | Dilution |
|---|---|---|---|---|---|---|
| TH | Polyclonal/Rabbit | N/A | Sigma | AB152 | WB | 1:1,000 |
| TH | Monoclonal/Mouse | N/A | EnCor | MCA-4H2 | WB, ELISA | 1:1,000 |
| TH | Polyclonal/Rabbit | N/A | EnCor | RPCA-TH | WB | 1:1,000 |
| TH | Polyclonal/Rabbit | Biotin | EnCor | RPCA-TH | ELISA | 1:6,000 |
| β3Tubulin | Polyclonal/Chicken | N/A | Aves | TUJ | WB | 1:1,000 |
| Chicken | Polyclonal/Rabbit | HRP | Sigma | A9046 | WB | 1:1,000 |
| Mouse | Polyclonal/Goat | IR-800 | Licor | 92632210 | WB | 1:15,000 |
| Rabbit | Polyclonal/Goat | IR-680 | Licor | 92568071 | WB | 1:15,000 |
| | | | | | Magnetic | |
| CD14 | Nanobeads | Magnetic | Biolegend | 480093 | Isolation | 20 uL/20M cells |
| Biotin | N/A (Avidin) | HRP | Vector Labs | A2004 | ELISA | 1:2,500 |

TABLE 5
Reagents and Materials

| | | Catalog | | |
|---|---|---|---|---|
| Reagent | Supplier | Number | Purpose | Concentration |
| EZ-Link Sulfo-NHS- | Thermo | A39257 | RPCA-TH biotinylation | 20-fold molar |
| LC-Biotin | Scientific | | | excess |
| Fat-free milk | Carnation | N/A | WB/ELISA | 1% or 5% |
| Clarity Western | Bio Rad | 1705061 | WB ECL | N/A |
| TMB Substrate | ThermoFisher | 34028 | ELISA | Stock |
| H2SO4 | Sigma | 339741 | ELISA | 2N |
| TritonX-100 | ThermoFisher | BP151-100 | MagneticIsolation | 1% |
| Tween-20 | ThermoFisher | MP1Tween201 | TBS-T | 0.2% |
| Protease Inhibitor | Millipore | 539191 | Cell lysis | 1x |
| DC Protein assay | Biorad | 5000112 | Protein assay | N/A |
| FBS | Gemini | 100-106 | Cell Culture | 10% or 5% |
| Horse Serum | Sigma | H1138-500ML | Cell Culture | 5% |
| Pen/Strep | ThermoFisher | 15-140-122 | Cell Culture | 1% |
| RPMI | Corning | 10-017 | Cell Culture | Stock |
| Immulon 4HBX | ThermoFisher | 3855 | ELISA | N/A |
| MojoSort Buffer 5x | Biolegend | 480017 | Magnetic cell isolation | 1x |
| Leucosep Tubes | Grenier BioOne | 227290P | PBMCisolation | N/A |

TABLE 6
Equipment

| Equipment | Supplier | Part Number | Purpose |
|---|---|---|---|
| Centrifuge | Eppendorf | 5424R | Cell lysate |
| | | | centrifugation |
| Centrifuge | Sorvall | ST8 | Cell culture |
| Magnet | Biolegend | 480019 | Magnetic Isolation |
| Plate reader | Biorad | iMark | WB/ELISA |
| Odyssey | Licor | Odyssey | WB |
| ChemiDoc+ | Biorad | ChemiDoc MP | WB |
| Mini-Protean Tetra | Biorad | 1658005 | WB |
| ELISA shaker | VWR | | ELISA incubations |
| Plate Washer | BioTek | ELX-405 | ELISA washes |

TABLE 7
Neuron culture reagents

| | | | |
|---|---|---|---|
| | | | Catalog |
| Chemical name | Concentration | Vendor | Number |

Dissociation Media

| NaCl | 116 | mM | Sigma- Aldrich | S7653 |
|---|---|---|---|---|
| NaHCo₃ | 26 | mM | Sigma- Aldrich | D6546 |
| NaH₂PO₄ | 2 | mM | Sigma- Aldrich | S9638 |
| D-glucose | 25 | mM | Sigma- Aldrich | G8769 |
| MgSO₄ | 1 | mM | Sigma- Aldrich | M7506 |
| Cysteine | 1.3 | mM | Sigma- Aldrich | C7352 |
| Papain | 400 units/ml | | Worthington | LS003127 |
| Kynurenic acid | 0.5 | mM | Sigma- Aldrich | K3375 |

| | | | |
|---|---|---|---|
| | | | Catalog |
| Chemical name | Concentration (%) | Vendor | Number |

Glia Media

| | | | |
|---|---|---|---|
| DMEM | 51.45 | Thermo Fisher | 11330032 |
| | | Scientific | |
| Fetal Bovine | 39.60 | Gemini | 100-106 |
| Serum | | | |
| Penicillin/ | 0.97 | Thermo Fisher | 15-140-122 |
| Streptomycin | | Scientific | |
| Glutamax | 0.97 | Thermo Fisher | 35050061 |
| 100X | | Scientific | |
| Insulin | 0.08 | Sigma-Aldrich | I5500 |
| (25 mg/ml | | | |
| stock) | | | |

Neuronal Media

| | | | |
|---|---|---|---|
| Neurobasal- | 96.9 | Thermo Fisher | 10888022 |
| A | | Scientific | |
| B27 Plus | 1.9 | Thermo Fisher | A3582801 |
| | | Scientific | |
| GDNF | 0.97 | Sigma-Aldrich | SRP3200 |
| Glutamax | 0.15 | Thermo Fisher | 35050061 |
| 100X | | Scientific | |
| Kynurenic | 0.08 | Sigma-Aldrich | K3375 |
| acid | | | |

Tyrosine Hydroxylase Recombinant Protein

Full length human TH protein was expressed from a synthetic codon optimized cDNA inserted into the EcoRI and SalI sites of the pET30a(+) vector, expressed in and purified from E. coli. The vector added a His-tag and other sequence to the N-terminus, a total of 5.7 kDa. Purification was performed using the His-tag on a nickel column. The TH sequence used in this study is the human tyrosine 3-monooxygenase isoform shown in Uniprot entry P07101-2 (see SEQ ID NO:1 below).

Cell Culture

Celllines: All cell cultures were conducted in 37° C. with 5% CO2. All reagents and supplies used for cell culture are outlined in Table 5. HEK293 cells do not express TH; therefore, they were used as a negative control group. The HEK293 cell were cultured as described previously^{1,2}, PC12 cells were cultured as described in Cartier et a. I 2010³. Briefly, HEK293 cells were grown in DMEM containing 10% FBS and 1% Pen/Strep until 70% confluence. PC12 cells express TH⁴; therefore, they were used as a positive control group. The PC12 were grown in DMEM containing 5% FBS, 5% Horse serum and 1% Pen/Strep.

Human macrophages: Primary human macrophages were cultured as described previously⁵. Briefly, PBMCs isolated as above were re-suspended in RPMI 1640 containing 1% Pen/Strep and 7.5% sterile-filtered, heat-inactivated autologous serum isolated from the donor's own blood, and plated in 24-well untreated polystyrene plates at 1 million PBMCs per well. Cells were washed to remove non-adherent cells (to retain only monocytes/macrophages) with incomplete RPMI 1640 and media replaced with complete media. Media was replaced at days 3 and 6 following culture, with lysis on Day 7 following culture.

Primary murine midbrain dopamine neurons: Midbrain dopamine neurons express TH (ref); therefore, they were used as a positive control group. Acutely dissociated mouse midbrains from 0-2 days old male and female pups were isolated and incubated in dissociation medium at 37° C. under continuous oxygenation for 90 minutes. Dissociated cells were pelleted by centrifugation at 1,500×g for 5 min and resuspended and triturated in glial medium. Cells were then plated on 12 mm coverslips coated with 0.1 mg/ml poly-D-lysine and 5 pg/ml laminin and maintained in neuronal media. Every 4 days, half the media was replaced with fresh media. The materials used for the preparation and maintenance of midbrain neuronal culture are outlined in Table 7.

Human Subjects

Blood samples from healthy subjects and Parkinson's diseases patients were obtained via an approved IRB protocol after obtaining informed consent, prior to January 2020 and before the first confirmed case of COVID-19 in the United States.

Blood samples from healthy subjects: Human blood samples were purchased from Lifesouth Community Blood Center, Gainesville, Fla. from August 2017 to January 2020 (prior to the first detected case of COVID-19 in the United States). The study was approved by the University of Florida's Institutional Review Board (IRB). According to Lifesouth rules and regulations, donors were healthy individuals aged 50-80 years-old of any gender, who were not known to have any blood borne pathogens (both self-reported, but also independently verified), and were never diagnosed with a blood disease, such as leukemia or bleeding disorders. In addition, none of the donors were using any medications for an infection or exhibiting signs/symptoms of infection, had a fever, had a positive test for viral infection in the previous 21 days, nor were they on any blood thinners, as determined from standard questionnaire.

Blood Samples from Parkinson's Disease Patients:

Blood samples were obtained from Parkinson's Disease (PD) patients and healthy age-matched controls at the University of Florida Center for Movements Disorders and Neurorestoration following an IRB-approved protocol. Patients and healthy controls were known to not have any recorded blood-borne pathogens or blood diseases, nor were they currently taking medications for infections or blood thinners according to their medical record.

Peripheral Blood Mononuclear Cell Isolation

Peripheral blood mononuclear cell express TH^{5,6}. As previously published⁶, whole blood was collected in K2EDTA vacutainer blood collection tubes (BD, 366643) and held at room temperature for up to 2 hours prior to PBMC isolation. Briefly, blood from healthy volunteers and Parkinson's patients was overlaid in Leucosep tubes (Table 6) for PBMC isolation, centrifuged for 20 minutes at 400 g with brakes turned off and acceleration set to minimum. PBMCs were collected from the interphase of Ficoll and PBS, transferred to a fresh 15 mL conical tube, resuspended in 8 mL sterile PBS and centrifuged for 10 minutes at 100 g, and repeated twice more. Cells were counted via hemacytometer with trypan blue exclusion of dead cells and density-adjusted for downstream applications.

Magnetic Monocyte Isolation

CD14+ monocytes express TH⁶. Primary CD14+ monocytes were isolated using Biolegend MojoSort Magnetic isolation kit (Cat #480017) per manufacturer's instructions.

Briefly, 20 million total PBMCs were counted, density adjusted to 1 million cells/uL, resuspended in MojoSort buffer, and incubated with TruStain Fc-block for 10 minutes at room temperature, followed by 1:10 anti-CD14 magnetic nanobeads for 15 minutes on ice. Following 2 washes with 2.5 mL ice-cold MojoSort buffer, cell pellet was resuspended in 2.5 mL MojoSort buffer and subject to three rounds of magnetic isolation per manufacturer's instructions. The resulting cell pellet was washed to remove remaining non-CD14+ cells and subject to cell lysis as detailed below.

Preparation of Cell Lysates

Buffer used for lysate preparation is given in Tables 5, 6 and 7. Adherent cells in culture were lifted using 0.02% EDTA in PBS, diluted with 5 volumes of PBS, and centrifuged at 100 g. Non-adherent cells (PC12) were centrifuged at 100 g for 5 minutes at room temperature, and cell pellets were washed 3 times with 5 volumes of sterile PBS. Primary macrophages and primary murine neuron cultures were washed thrice with ice-cold PBS, on ice. Cell pellets and adherent primary cells were then lysed in ice-cold lysis buffer (10 mM NaCl, 10% Glycerol (v/v), 1 mM EDTA, 1 mM EGTA, and HEPES 20 mM, pH 7.6), with Triton X-100 added to a final concentration of 1%, containing 1× protease inhibitor cocktail (Millipore-Sigma, Cat 539131) for one hour at 4° C. with rotation. Resulting lysate was centrifuged at 12,000 g for 15 minutes at 4° C. Supernatant was set aside for protein quantification by Lowry Assay (Biorad, 5000112) and the remainder was stored at −80° C. until use for downstream assays.

Western Blot

Reagents, antibodies and equipment are given in Tables 5, 6 and 7. Samples of PC12 lysate (5 ug) and purified TH peptide (60 ng) were incubated in Laemmli sample buffer containing 10% beta-mercaptoethanol at 37° C. for 30 minutes, separated by SDS-PAGE on 10% bis/polyacrylamide gels, and transferred to nitrocellulose membranes. After first blocking for 1 hour in TBS-T (50 mM Tris-HCl, 150 mM NaCl, and 0.1% Tween 20) containing 5% dry milk (blocking buffer), then incubated with primary antibody against TH (Table 4) overnight at 4° C. Membranes were then incubated with an appropriate secondary antibody (Table 4) for 1 hour at room temperature with agitation. Following all antibody steps, membranes were washed three times for 5 minutes each using TBS-T. Loading controls (beta3-tubulin, Aves, TUJ) were detected following 1 hour blocking and 1 hour antibody incubations (primary and HRP-secondary, Table 4), followed by ECL detection. TH was visualized using Licor Odyssey and tubulin was visualized using Bio-Rad ChemiDoc+(Table 6).

RPCA-TH Biotinylation

EZ-Link Sulfo-NHS-LC-Biotin (A39257, Thermo Scientific) at 20-fold molar biotin reagent excess using the manufacturers protocol. 1 mg of antibody was concentrated to 2 mg/mL, brought to pH 8.0 with sodium bicarbonate and reacted with reagent for 1 hour at room temperature. The conjugate was purified by gel filtration on a Biorad 10DG column (cat 732-2010).

ELISA for TH

Antibodies used for ELISA are described in Table 4. Ten lanes of an Immulon 4 HBX High-Binding 96 well plate were coated with 100 uL per well of 1:1,000 dilution of mouse anti-TH (MCA-4H2) in coating buffer (28.3 mM Na₂CO₃, 71.42 mM NaHCO₃, pH 9.6) for 20 hours at 4° C. Edge lanes 1 and 12 were left empty. Wells were blocked with 5% fat free milk in 1×TBS (pH 7.4) for 1 hour at room temperature on an orbital shaker set to 90 rpm. To produce a standard curve, two standard curve lanes were generated, with six serial dilutions, beginning at 10 ng/mL and 1 ng/mL in TBS-T containing 1% fat free milk (with the last well in each standard curve lane left with incubation buffer only as a blank. Remaining wells were incubated in duplicate with lysates from cells of interest. Incubation was completed for 20 hours at 4° C. on an ELISA shaker set to 475 rpm.

After each well was washed and aspirated 6 times with TBS-T, anti-TH rabbit (EnCor, RPCA-TH) conjugated to biotin was dilute 1:6,000 from a stock concentration of 1.65 mg/mL in TBS-T with 1% fat-free milk and incubated for 1 hour at room temperature at 425 rpm. 100 uL Avidin-HRP (Vector labs, A-2004), dilute 1:2,500 in TBS-T with 1% fat-free milk, was added to each well following washes, as described above, and incubated for 1 hour at room temperature at 425 rpm. Following final washes, 150 uL room temperature TMB-ELISA reagent (Thermo Fisher, 34028) was added to each well. The reaction was allowed to continue for 20 minutes, protected from light, and stopped by addition of 50 uL 2N H₂SO₄. The plate was immediately read at 450 nm.

Duplicate standard and sample wells were averaged, and background-subtracted based on blank wells. The concentration of TH for each experimental group was calculated using a quadratic curve equation calculated in Graphpad Prism 8, then normalized to total protein concentration per sample as calculated using Lowry Assay. Final TH values shown are presented as pg TH/mg total protein after multiplication of the nanogram TH value by 1,000 to show TH as picogram TH/milligram total protein.

### Results

Novel Quantitative ELISA Successfully and Reproducibly Detects Tyrosine Hydroxylase. A Novel Quantitative

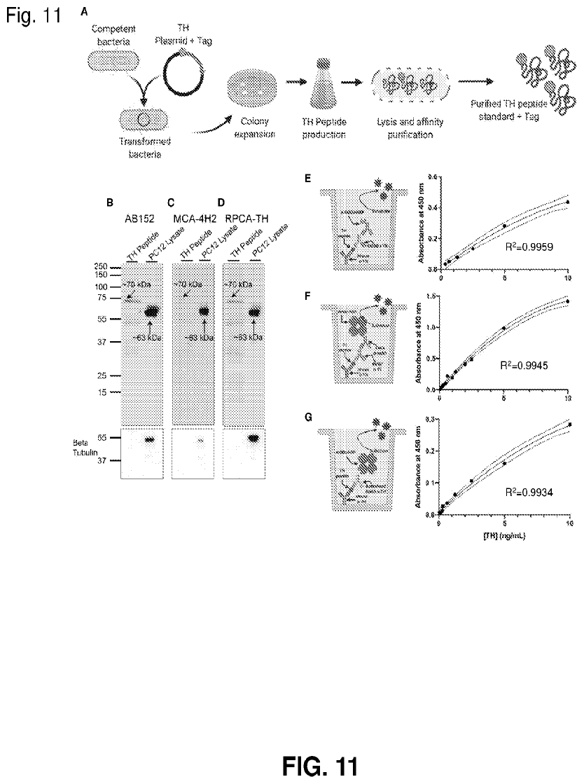

ELISA for tyrosine hydroxylase (TH) depends on the availability of pure TH and high-quality antibodies against TH, preferably generated in two distinct host species. The fully length human TH was first expressed and purified using a codon optimized synthetic DNA based on Uniprot entry P07101 designed for expression using the pET30a(+) vector in E. coli. which adds an N-terminal His-tag. The construct was expressed using standard procedures and purified by Nickel affinity chromatography using the vector derived His-tag and used to generate antibodies in rabbits and mice (FIG. 11A). A panel of antibodies were generated and appropriate quality assessment was performed by ELISA, western blotting and cell and tissue staining. A mouse monoclonal antibody MCA-4H2 and a rabbit polyclonal RPCA-TH, were selected as capture and detection antibodies, respectively. The identity of the TH construct was confirmed via western blot analysis using a commercially available TH antibody (AB152, Millipore-Sigma)⁷⁻¹⁰. The TH recombinant protein band identity was compared to TH expression in PC12 cells (FIG. 11B). As predicted, PC12 lysate shows a single TH band at ˜63 kDa, with a corresponding band for the purified TH peptide at ˜70 kDa. Observed difference in molecular weight between TH expressed in PC12 cells and the TH recombinant protein is consistent with the 5.7 kDa tag added to the recombinant TH protein N-terminus. Beta3 tubulin (TUJ, Aves) is shown to indicate that cell lysate was loaded in PC12 lanes but not in TH recombinant lanes. Both novel TH antibodies detected TH both in positive controls and recombinant TH conditions using Western Blot analysis. Both MCA-4H2 and RPCA-TH reliably detect both recombinant TH and native TH in PC12 lysate (FIG. 11C, D), confirming again both the identity of the purified TH peptide as well as specificity of both antibodies selected for ELISA development.

To quantify TH expression in control conditions, a standard sandwich ELISA approach (FIG. 11E) was first attempted, in which MCA-4H2 was used as the capture antibody, followed by incubation with recombinant TH, then RPCA-TH as detection antibody. Enzyme-based detection was accomplished by addition of HRP-conjugated secondary (goat anti-rabbit HRP, Vector, BA1000). While this functioned and reliably quantified TH, this initial version of the assay produced a lower detection threshold of 125 pg/mL TH. The sensitivity of the assay was sought to be increased by addition of a biotin-avidin amplification step (Avidin-HRP, Vector, A2004) (FIG. 11F), providing improved lower threshold of 62.5 pg/mL. A further refinement was the biotinylation of the rabbit detection antibody using Sulfo-NHS-LC-biotin (Thermo Scientific A39257) which improved sensitivity further by reducing background and producing a lower-threshold of detection at 15 pg/mL. Both novel TH antibodies are available commercially from EnCor Biotechnology Inc.

TH ELISA Reliably Quantifies TH in PC12 Cells, Human Macrophages, and Cultured Murine Dopaminergic Neurons.

Having established a method with a suitably low detection threshold, the novel TH ELISA was tested on cell homogenates prepared from PC12 cells (postive control group), HEK293 cells (negative control group), cultured primary human macrophages derived from whole blood samples from healthy donors (experimental group), and cultured primary midbrain dopamine neurons prepared from PNDO-PND3 mouse pups (positive control group). PC12 cells are known to express high levels of TH⁴, while HEK293 serve as negative control¹¹. Cultured midbrain dopamine neurons are known to express TH as the rate limiting enzyme for dopamine synthesis², cultured human monocyte-derived-macrophages express TH protein and mRNA^{5,12}.

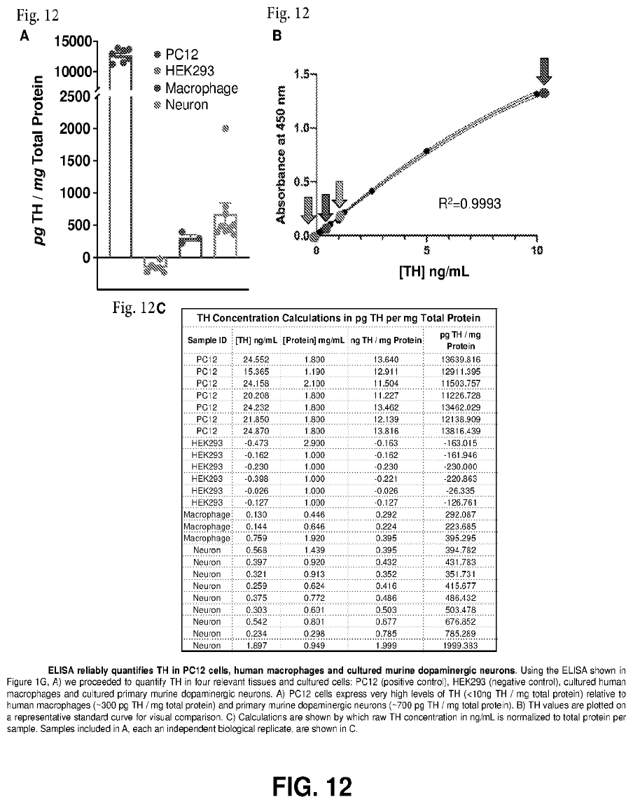

TH expression is shown as unit TH (picogram or nanogram) per mg total protein, as determined by the Lowry Assay. PC12 homogenate provided a reliable positive control expressing high levels of TH (<10 ng TH/mg total protein), while HEK293 homogenate showed no detectable levels of TH, in at least 6 independent replicates. As anticipated, cultured dopamine neurons from postnatal mice showed greater TH concentrations (˜700 pg TH/mg total protein) than cultured human macrophages (˜300 pg TH/mg total protein) (FIG. 12A), suggesting this assay is applicable to cell and tissue samples derived from human and murine specimens, paving the way for use in translational and preclinical studies involving TH protein expression levels. Visual representation of relative TH expression in PC12, HEK293, macrophage and primary neuron homogenates are plotted on a representative standard curve (FIG. 12B). Raw values [TH] in ng/mL calculated from absorbance are shown in FIG. 12C, alongside each sample ID. Raw TH concentration was divided by [Protein], then multiplied by 1,000 to produce values in pg TH/mg total protein (FIG. 12C).

Purified Monocytes Isolated from Blood of Parkinson's Disease Patients Show Increased TH Concentration Relative to Healthy Controls.

So far, the ELISA was used to reliably quantify TH in multiple well-established model systems, which are known to express TH-PC12 cells, rodent dopamine neurons and cultured macrophages. Next, the assay was tested toward measuring TH expression in clinical samples of freshly isolated human monocytes. Peripheral immune cells are exposed to picomolar to nanomolar concentrations of dopamine in circulation and in organs such as thymus, pancreas, bone marrow, heart, spleen, kidney, lung, throughout the gut and with micromolar concentration in the carotid body¹²⁻¹⁶. Human myeloid cells, such as monocytes and macrophages express all subtypes of dopamine receptors (DRs), as well as dopamine transporter (DAT), vesicular monoamine transporter (VMAT), and aromatic amino-acid decarboxylase (AADC)¹⁷⁻²⁰, and they store and synthesize dopamine²¹⁻²⁵. Given that human monocyte derived macrophages express TH^{5, 12, 19, 20}, as do circulating monocytes⁶, the choice was made to investigate whether peripheral monocytes express altered TH in Parkinson's disease patients, a disease state in which monoamine signaling is globally affected¹². Since both norepinephrine and dopamine require TH as the rate limiting enzyme for synthesis, it was predicted that in Parkinson's disease where brain monoamines and peripheral dopamine and norepinephrine are reduced, TH would be similarly reduced.

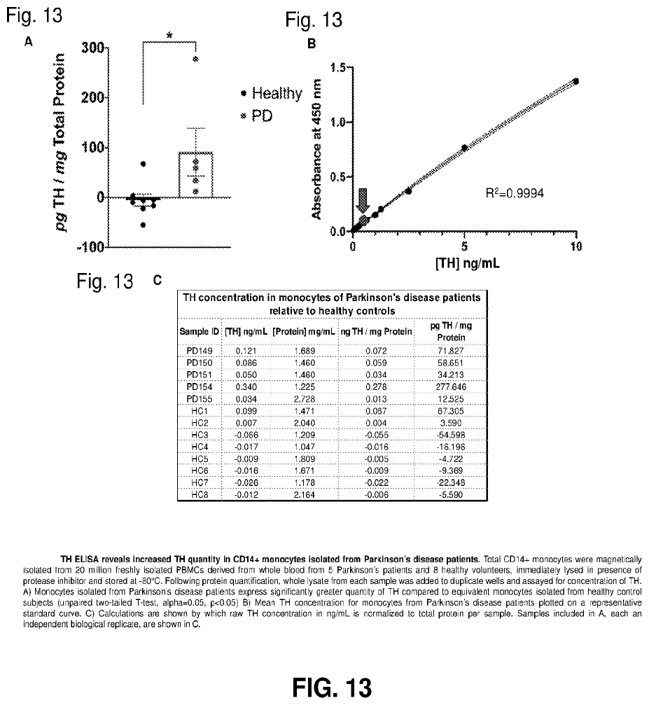

In order to assess whether TH levels were altered in peripheral monocytes of patients with Parkinson's disease, 5 Parkinson's patients and 8 healthy control subjects were recruited via an approved IRB protocol. Total monocytes for each subject were isolated from 20 million total PBMCs using anti-CD14 magnetic isolation per manufacturer's instructions (Mojosort, Biolegend, 480093). Purified monocytes were immediately lysed and assayed via the ELISA for TH concentration following total protein quantification. Of eight healthy control samples included, only two registered TH concentrations above the detection threshold. By contrast, all Parkinson's disease patients recruited for this study show clear positive TH values, indicating that Parkinson's disease patients' monocytes express significantly more TH protein relative to healthy control subjects (P<0.05). See FIG. 13.

### Sequence Information

### SEQ ID NO:1

```
                10 20 30 40
MPTPDATTPQ AKGFRRAVSE LDAKQAEAIM GAPGPSLTGS
                50 60 70 80
PWPGTAAPAA SYTPTPRSPR FIGRRQSLIE DARKEREAAV
                90 100 110 120
AAAAAAVPSE PGDPLEAVAF EENEGKAVLN LLFSPRATKP
              130 140 150 160
SALSRAVKVF ETFEAKIHHL ETRPAQRPRA GGPHLEYFVR
              170 180 190 200
LEVRRGDLAA LLSGVRQVSE DVRSPAGPKV PWFPRKVSEL
              210 220 230 240
DKCHHLVTKF DPDLDLDHPG FSDQVYRQRR KLIAEIAFQY
              250 260 270 280
RHGDPIPRVE YTAEEIATWK EVYTTLKGLY ATHACGEHLE
              290 300 310 320
AFALLERFSG YREDNIPQLE DVSRFLKERT GFQLRPVAGL
              330 340 350 360
LSARDFLASL AFRVFQCTQY IREASSPMHS PEPDCCHELL
              370 380 390 400
GHVPMIADRT FAQFSQDIGL ASLGASDEEI EKLSTLYWFT
              410 420 430 440
VEFGLCKQNG EVKAYGAGLL SSYGELLHCL SEEPEIRAFD
              450 460 470 480
PEAAAVQPYQ DQTYQSVYFV SESFSDAKDK LRSYASRIQR
              490 500 510 520
PFSVKFDPYT LAIDVLDSPQ AVRRSLEGVQ DELDTLAHAL
SAIG
```

### REFERENCES FOR EXAMPLE 2

1 Goodwin, J. S. et al. Amphetamine and methamphetamine differentially affect dopamine transporters in vitro and in vivo. J Biol Chem 284, 2978-2989, doi:10.1074/jbc. M805298200 (2009).
2 Saha, K. et al. Intracellular methamphetamine prevents the dopamine-induced enhancement of neuronal firing. J Biol Chem 289, 22246-22257, doi:10.1074/jbc. M114.563056 (2014).
3 Cartier, E. A. et al. A biochemical and functional protein complex involving dopamine synthesis and transport into synaptic vesicles. J Biol Chem 285, 1957-1966, doi:10.1074/jbc. M109.054510 (2010).
4 Greene, L. A. & Tischler, A. S. Establishment of a noradrenergic clonal line of rat adrenal pheochromocytoma cells which respond to nerve growth factor. Proc Natl Acad Sci USA 73, 2424-2428, doi:10.1073/pnas.73.7.2424 (1976).
5 Mackie, P. et al. The dopamine transporter: An unrecognized nexus for dysfunctional peripheral immunity and signaling in Parkinson's Disease. Brain Behav Immun 70, 21-35, doi:10.1016/j.bbi.2018.03.020 (2018).
6 Gopinath, A. et al. A novel approach to study markers of dopamine signaling in peripheral immune cells. J Immunol Methods 476, 112686, doi:10.1016/j.jim.2019.112686 (2020).
7 Giguere, N. et al. Increased vulnerability of nigral dopamine neurons after expansion of their axonal arborization size through D2 dopamine receptor conditional knockout. PLoS Genet 15, e1008352, doi:10.1371/journal.pgen.1008352 (2019).
8 Colon-Perez, L. M. et al. Functional connectivity, behavioral and dopaminergic alterations 24 hours following acute exposure to synthetic bath salt drug methylenedioxypyrovalerone. Neuropharmacology 137, 178-193, doi:10.1016/j.neuropharm.2018.04.031 (2018).
9 Contini, M. & Raviola, E. GABAergic synapses made by a retinal dopaminergic neuron. Proc Natl Acad Sci USA 100, 1358-1363, doi:10.1073/pnas.0337681100 (2003).
10 Feinstein, P., Bozza, T., Rodriguez, I., Vassalli, A. & Mombaerts, P. Axon guidance of mouse olfactory sensory neurons by odorant receptors and the beta2 adrenergic receptor. Cell 117, 833-846, doi:10.1016/j.cell.2004.05.013 (2004).
11 Graham, F. L., Smiley, J., Russell, W. C. & Nairn, R. Characteristics of a human cell line transformed by DNA from human adenovirus type 5. J Gen Virol 36, 59-74, doi:10.1099/0022-1317-36-1-59 (1977).
12 Matt, S. M. & Gaskill, P. J. Where Is Dopamine and how do Immune Cells See it?: Dopamine-Mediated Immune Cell Function in Health and Disease. J Neuroimmune Pharmacol, doi:10.1007/s11481-019-09851-4 (2019).
13 Goldstein, D. S., Eisenhofer, G. & Kopin, I. J. Sources and significance of plasma levels of catechols and their metabolites in humans. J Pharmacol Exp Ther 305, 800-811, doi:10.1124/jpet.103.049270 (2003).
14 Eisenhofer, G. et al. Biochemical and clinical manifestations of dopamine-producing paragangliomas: utility of plasma methoxytyramine. J Clin Endocrinol Metab 90, 2068-2075, doi:10.1210/jc.2004-2025 (2005).
15 Goldstein, D. S., Holmes, C. & Axelrod, F. B. Plasma catechols in familial dysautonomia: a long-term follow-up study. Neurochem Res 33, 1889-1893, doi:10.1007/s11064-008-9662-4 (2008).
16 Goldstein, D. S. et al. Sources and physiological significance of plasma dopamine sulfate. J Clin Endocrinol Metab 84, 2523-2531, doi:10.1210/jcem.84.7.5864 (1999).
17 Coley, J. S., Calderon, T. M., Gaskill, P. J., Eugenin, E. A. & Berman, J. W. Dopamine increases CD14+CD16+ monocyte migration and adhesion in the context of substance abuse and HIV neuropathogenesis. PLoS One 10, e0117450, doi:10.1371/journal.pone.0117450 (2015).
18 Eugenin, E. A., Gaskill, P. J. & Berman, J. W. Tunneling nanotubes (TNT) are induced by HIV-infection of macrophages: a potential mechanism for intercellular HIV trafficking. Cell Immunol 254, 142-148, doi:10.1016/j.cellimm.2008.08.005 (2009).
19 Nolan, R. & Gaskill, P. J. The role of catecholamines in HIV neuropathogenesis. Brain Res 1702, 54-73, doi:10.1016/j.brainres.2018.04.030 (2019).
20 Nolan, R. A., Muir, R., Runner, K., Haddad, E. K. & Gaskill, P. J. Role of Macrophage Dopamine Receptors in Mediating Cytokine Production: Implications for Neuroinflammation in the Context of HIV-Associated Neurocognitive Disorders. J Neuroimmune Pharmacol 14, 134-156, doi:10.1007/s11481-018-9825-2 (2019).
21 Cosentino, M. et al. HPLC-ED measurement of endogenous catecholamines in human immune cells and hematopoietic cell lines. Life Sci 68, 283-295, doi:10.1016/s0024-3205(00)00937-1 (2000).
22 Flierd, M. A. et al. Upregulation of phagocyte-derived catecholamines augments the acute inflammatory response. PLoS One 4, e4414, doi:10.1371/journal.pone.0004414 (2009).
23 Marino, F. et al. Endogenous catecholamine synthesis, metabolism storage, and uptake in human peripheral blood mononuclear cells. Exp Hematol 27, 489-495 (1999).
24 Scheiermann, C., Frenette, P. S. & Hidalgo, A. Regulation of leucocyte homeostasis in the circulation. Cardiovasc Res 107, 340-351, doi:10.1093/cvr/cvv099 (2015).
25 Shi, C. & Pamer, E. G. Monocyte recruitment during infection and inflammation. Nat Rev Immunol 11, 762-774, doi:10.1038/nri3070 (2011).

What is claimed is:

1. A method for detecting at least one mediator of dopamine transmission, the method comprising:
obtaining a cell suspension comprising a plurality of peripheral blood mononuclear cells from a first subject;
staining the plurality of peripheral blood mononuclear cells with a fluorescent marking compound to produce a stained cell suspension;
detecting a level of the at least one mediator of dopamine transmission in at least a portion of the stained cell suspension by passing the portion through a flow cytometer.

2. The method, according to claim 1, wherein the at least one mediator of dopamine transmission is selected from the group consisting of dopamine transporter, tyrosine hydroxylase, and combinations thereof.

3. The method, according to claim 1 or 2, wherein the cell suspension is cryopreserved prior to detecting the level of the at least one mediator of dopamine transmission.

4. The method according to any of claims 1-3, wherein the cell suspension is cryopreserved with CryoStor10.

5. The method, according to any of claims 1-4, further comprising comparing the level of the at least one mediator of dopamine transmission with a control level, wherein the control level is a level of the at least one mediator of dopamine transmission in a cell suspension peripheral blood mononuclear cells from a healthy subject.

6. The method, according to any of claims 1-5, wherein the healthy subject is a human, and wherein the average level is about 12% PBMCs.

7. The method, according to claim 6, further comprising diagnosing a disease based at least in part on comparing the level with the average level.

8. The method, according to claim 7, wherein the disease is a neurodegenerative disease.

9. The method, according to claim 8, wherein the neurodegenerative disease is Parkinson's disease.

10. The method according to claim 9, wherein when Parkinson's disease is diagnosed, administering a Parkinson's therapy to the first subject.

11. The method of claim 10, wherein the Parkinson's therapy comprises a therapeutically effective amount of a composition comprising levodopa, carbidopa, a dopamine agonist, a MAO B inhibitor, a COMT inhibitor, an anticholinergic, or amantadine, or a combination thereof.

12. A method comprising detecting a level of a dopamine transporter (DAT) and/or a tyrosine hydroxylase (TH) in a biosample from a subject, wherein the biosample comprises a homogenate of peripheral monocytes from the subject and wherein detecting comprises conducting an ELISA assay.

13. The method of claim 12, further comprising determining that the subject has a neurodegenerative disease if the level of DAT and/or TH is 10% or higher than a level of a control biosample comprising a homogenate of peripheral monocytes from a healthy subject.

14. The method of any of claims 12-13, wherein detecting comprises binding an antibody to DAT or TH in the biosample.

15. The method of claim 16, wherein the antibody is biotinylated.

16. The method, according to any of claims 13-15, wherein the disease is a neurodegenerative disease.

17. The method, according to claim 16, wherein the neurodegenerative disease is Parkinson's disease.

18. The method according to claim 17, wherein when Parkinson's disease is diagnosed, administering a Parkinson's therapy to the first subject.

19. The method of claim 18, wherein the Parkinson's therapy comprises a therapeutically effective amount of a composition comprising levodopa, carbidopa, a dopamine agonist, a MAO B inhibitor, a COMT inhibitor, an anticholinergic, or amantadine, or a combination thereof.

20. A method comprising detecting a level of tyrosine hydroxylase (TH) in a biosample from a subject, wherein the biosample comprises a homogenate of peripheral monocytes from the subject; and comparing the level of TH in the biosample with a control biosample comprising a homogenate of peripheral monocytes from a healthy subject.

21. The method of claim 20, further comprising administering a Parkinson's therapy if the level of TH is 10% or higher than a level of the control biosample.

22. The method of any of claim 20 or 21, wherein detecting comprises binding an antibody to TH in the biosample.

23. The method of claim 22, wherein the antibody is biotinylated.

24. The method of any of claims 21-23, wherein the Parkinson's therapy comprises a therapeutically effective amount of a composition comprising levodopa, carbidopa, a dopamine agonist, a MAO B inhibitor, a COMT inhibitor, an anticholinergic, or amantadine, or a combination thereof.

25. A kit comprising tools, reagents and equipment that enable immunoassay (such as antibodies, aptamers) and/or miRNA amplification (e.g. PCR, RCA) based detection of DAT or TH at either point of care (POC) or core lab test setting using cell based biosample.
